(19) United States
(12) Patent Application Publication
(10) Pub. No.: US 2026/0224490 A1
(43) Pub. Date: Aug. 6, 2026

(54) CO-CRYSTALS WITH THIN-FILM FREEZE-DRYING PROCESS TO ENHANCE DELIVERY

(71) Applicant: BOARD OF REGENTS, THE UNIVERSITY OF TEXAS SYSTEM, Austin, TX (US)

(72) Inventors: Robert O. WILLIAMS, III, Austin, TX (US); Jay I. PETERS, San Antonio, TX (US); Tuangrat PRAPHAWATVET, Austin, TX (US); Sawittree SAHAKIJPIJARN, Austin, TX (US); Chaeho MOON, Austin, TX (US)

(21) Appl. No.: 19/147,310

(22) PCT Filed: Jan. 11, 2024
(86) PCT No.: PCT/US2024/011210

§ 371 Date: Jul. 10, 2025

Related U.S. Application Data
(60) Provisional application No. 63/479,457, filed on Jan. 11, 2023.

(51) Int. Cl. A61K 9/14; A61K 9/00; A61K 31/365; A61K 31/4412; A61K 47/18; A61K 47/26

(52) U.S. Cl. CPC A61K 9/145; A61K 9/0075; A61K 9/143; A61K 31/365; A61K 31/4412; A61K 47/183; A61K 47/26

(57) ABSTRACT

The present disclosure provides co-crystals of an active pharmaceutical ingredient and a co-former. The co-former may be either an excipient or a second active pharmaceutical ingredient. These particular co-crystals may be made through thin-film freeze-drying methods. Pharmaceutical compositions comprising these co-crystals may be used in the treatment of a disease or disorder.

This application claims the benefit of priority to U.S. Provisional Application No. 63/479,457, filed on Jan. 11, 2023, the entire contents of which are hereby incorporated by reference.

### BACKGROUND

### I. Field

The present disclosure relates generally to the field of pharmaceuticals and pharmaceutical manufacture. More particularly, it concerns compositions and methods of making co-crystals using thin-film freeze-drying manufacturing techniques.

### II. Description of Related Art

Pulmonary drug delivery has advanced significantly over the last decade. Orally inhaled products have been developed as delivery systems for both local treatments of lung diseases (e.g., chronic obstructive pulmonary disease, asthma, tuberculosis) and the systemic treatment of several diseases such as diabetes (Pfutzner and Forst, 2005), measles (Griffin, 2014), Parkinson's disease (LeWitt et al., 2018), schizophrenia (Kristin et al., 2016), and influenza (Silveira et al., 2016). The dry powder inhaler (DPI) is considered the most promising dosage form, as opposed to pressurized metered-dose inhalers or nebulizers. DPIs provide several advantages, including ease of operation and portability. In addition, they do not require propellants, they allow for relatively low-cost devices, and they offer enhanced stability of the active component as a result of their dry state (Carpenter et al., 1997).

Oral pirfenidone has high oral doses which can cause serious side effects, notably gastrointestinal disorders, photosensitivity reactions and drug-induced liver disorders. Pirfenidone has a narrow therapeutic index that has to be monitored by serum concentration in patients during a treatment. Moreover, oral pirfenidone exhibits difficult drug management. Bioavailability of pirfenidone can be decreased by food; yet fasted patients have a higher incidence of adverse reaction compared with fed patients. Dose adjustments then have to be made in patients who cannot tolerate said side effects. Therefore, methods for making pharmaceutical compositions which are administered via routes other than oral administration are desired.

The development of inhaled products must address several physical difficulties to achieve effective drug delivery. The aerodynamic diameter of drug particles must be between 1 μm and 5 μm to maximize the probability of drug particles from a DPI reaching the lower respiratory tract (Prime et al., 1997). However, such micronized drug particles have high forces of cohesiveness and a tendency to agglomerate, which results in poor flowability, poor aerosolization properties, and high dose variability (Chan and Chew, 2003). For at least these reasons, there is an unmet need for methods of preparing inhalable pharmaceutical compositions with improved properties.

An inhaled pirfenidone powder which exhibits high aerodynamic properties may achieve pulmonary delivery. Dry powder for inhalation of pirfenidone can provide therapeutic effects at a lower delivered dose, resulting in a reduction of undesirable side effects. However, methods for making inhalable co-crystals of pirfenidone and other drugs with low-aerosol properties, which may be produced by thin-film freeze-drying process to further improve aerodynamic performance, has not been established.

### SUMMARY

The present disclosure provides pharmaceutical compositions comprising one or more co-crystals that may be prepared using thin-film freeze-drying methods.

In some aspects, the present disclosure provides a method of providing a pharmaceutical composition comprising:
(A) admixing a drug solution comprising an active pharmaceutical ingredient and a co-former to form a co-crystal solution;
(B) admixing the co-crystal solution with an excipient to form a precursor pharmaceutical composition;
(C) depositing the precursor pharmaceutical composition onto a surface, wherein the surface is at a surface temperature below the freezing point of the precursor pharmaceutical composition to produce composite drug particles; and
(D) collecting the composite drug particles from the surface to thereby provide the pharmaceutical composition.

In some aspects, the co-former is added a co-former solution comprising the co-former and a co-former solution solvent. In some embodiments, the co-former comprises an acid group selected from either a phosphoric acid group, a sulfuric acid group, or carboxylic acid group. In some embodiments, the co-former comprises a carboxylic acid group. In some aspects, the co-former comprises one or two carboxylic acid groups.

In some embodiments, the co-former is an aliphatic carboxylic acid. In certain embodiments, the co-former is an aliphatic carboxylic acid having from 1-8 carbon atoms. In certain embodiments, the co-former is an aliphatic carboxylic acid having from 3-8 carbon atoms. In further embodiments, the co-former is an aliphatic carboxylic acid having from 3-6 carbon atoms. In yet further embodiments, the co-former is an aliphatic carboxylic acid having 1 carbon atom.

In some embodiments, the co-former is formic acid. In some embodiments, the co-former is an aliphatic carboxylic acid having 2 carbon atoms, such as acetic acid or trifluoroacetic acid. In some embodiments, the co-former is an aliphatic carboxylic acid having 4 carbon atoms such as succinic acid or fumaric acid. In certain embodiments, the co-former is succinic acid. In other embodiments, the co-former is fumaric acid.

In some embodiments, the co-former is an aliphatic carboxylic acid having six carbon atoms, such as citric acid. In some embodiments, the co-former is a sulfuric acid group or phosphoric acid group. In some embodiments, the inorganic acid is phosphoric acid. In some embodiments, the co-former comprises a basic group, such as an aliphatic group comprising a basic group. In some embodiments, the aliphatic group comprising a basic group has from 1 to 8 carbon atoms. In some embodiments, the aliphatic group comprising a basic group has from 3 to 8 carbon atoms.

In some embodiments, the basic group is an amine group, such as a primary amine group, a secondary amine group, a tertiary amine group, or a quarternary amine group. In some embodiments, the co-former is a second active pharmaceutical ingredient. In some embodiments, the second active pharmaceutical ingredient is different from the active pharmaceutical ingredient. In some embodiments, the second active pharmaceutical ingredient comprises an acidic group, such as a carboxylic acid or mycophenolic acid. In some embodiments, the second active pharmaceutical ingredient comprises a basic group, such as an amine group.

In some aspects, the method comprises using a molar ratio of the active pharmaceutical ingredient and co-former from about 1:5 to about 10:1. In some aspects, the molar ratio is from about 1:2 to about 5:1. In some aspects, the molar ratio is from about 1:2 to about 2:1. In some aspects, the molar ratio is about 1:1. In other aspects, the molar ratio is about 2:1.

In some embodiments, the co-former solution solvent is a protic solvent, such as water. In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

In some embodiments, the active pharmaceutical ingredient is an anti-inflammatory agent, such as an anti-fibrotic agent. In some embodiments, the anti-inflammatory agent is pirfenidone. In some embodiments, the drug solution comprises an active pharmaceutical ingredient and a drug solution solvent, such as an organic solvent. In some embodiments, the drug solution solvent is a polar aprotic solvent. In some embodiments, the drug solution solvent is a C1-C8 cyanoalkyl. In some embodiments, the drug solution solvent is acetonitrile.

In some embodiments, the excipient is added as an excipient solution comprising an excipient and an excipient solution solvent. In some embodiments, the excipient is a sugar or sugar alcohol. In some embodiments, the excipient is a sugar, such as a polysaccharide. In some embodiments, the excipient is a disaccharide, such as trehalose or lactose. In some embodiments, the excipient is lactose. In some embodiments, the excipient is lactose that has been milled with a particle size.

In some embodiments, the particle size has a D₅₀ as measured by laser diffraction from about 10 μm to about 150 μm. In some embodiments, the particle size has a D₅₀ as measured by laser diffraction from about 50 μm to about 125 μm. In some embodiments, the particle size has a D₅₀ as measured by laser diffraction from about 75 μm to about 100 μm.

In some embodiments, the excipient is a sugar alcohol, such as mannitol. In some embodiments, the excipient is an amino acid or a peptide. In certain embodiments, the excipient is an amino acid. In some embodiments, the excipient is a hydrophobic amino acid, such as an aliphatic hydrophobic amino acid. In some embodiments, the excipient is leucine. In some embodiments, the excipient is a polar amino acid, such as lysine. In some embodiments, the excipient is glycine.

In some embodiments, the excipient solution solvent is a protic solvent, such as water. In some embodiments, the excipient solution solvent is an organic solvent. In some embodiments, the excipient solution solvent is a polar protic solvent. In some embodiments, the solvent is a C1-C8 alcohol, such as t-butanol.

In some embodiments, the precursor pharmaceutical composition comprises a solid content from about 0.05% w/v to about 10% w/v. In some embodiments, the solid content is from about 0.1% w/v to about 5% w/v. In certain embodiments, the solid content is from about 0.25% w/v to about 2.5% w/v. In certain embodiments, the solid content is from about 0.25% w/v to about 1% w/v. In certain embodiments, the solid content is about 0.5% w/v.

In some aspects, the method further comprises depositing a discrete amount of the precursor pharmaceutical composition onto the surface. In some embodiments, the discrete amount is from about 0.5 μL to about 40 μL. In certain embodiments, the discrete amount is from about 1 μL to about 30 μL. In certain embodiments, the discrete amount is from about 10 μL to about 20 μL. In certain embodiments, the discrete amount is 15 μL.

In some aspects, the method further comprises depositing the precursor pharmaceutical composition onto the surface from a fixed height. In some embodiments, the fixed height is from about 1 cm to about 25 cm. In certain embodiments, the fixed height is from about 5 cm to about 20 cm. In certain embodiments, the fixed height is from about 7.5 cm to about 15 cm. In yet further embodiments, the fixed height is from about 7.5 cm to about 12.5 cm. In certain embodiments, the fixed heigh is about 10 cm.

In some embodiments, the surface temperature is less than −10° C. In other embodiments, the surface temperature is from about −10° C. to about −200° C. In certain embodiments, the surface temperature is from about −50° C. to about −175° C. In certain embodiments, the surface temperature is from about −100° C. to about −150° C. In certain embodiments, the surface temperature is about −120° C.

In some embodiments, the method further comprises drying the pharmaceutical composition. In some embodiments, the drying process comprises lyophilization. In some embodiments, the drying process comprises two drying cycles. In some embodiments, the first drying cycle comprises drying at a first temperature from about 0° C. to about −120° C. In some embodiments, the first temperature is a temperature from about −10° C. to about −80° C. In certain embodiments, the first temperature is a temperature from about −20° C. to about −60° C.

In some embodiments, the first drying cycle comprises drying at a reduced pressure. In some embodiments, the reduced pressure is a first pressure from about 10 mTorr to about 500 mTorr. In certain embodiments, the first pressure is from about 25 mTorr to about 250 mTorr. In some embodiments, the first pressure is from about 50 mTorr to about 150 mTorr.

In some embodiments, the second drying cycle comprises drying at a second temperature from about 0° C. to about 80° C. In some embodiments, the second temperature is a temperature from about 10° C. to about 60° C. In certain embodiments, the second temperature is a temperature from about 20° C. to about 50° C. In some embodiments, the second drying cycle comprises drying at a reduced pressure. In some embodiments, the reduced pressure is a second pressure from about 10 mTorr to about 500 mTorr. In some embodiments, the second pressure is from about 25 mTorr to about 250 mTorr. In certain embodiments, the second pressure is from about 50 mTorr to about 150 mTorr.

In some embodiments, the pharmaceutical composition comprises particles of the co-crystal having a size from about 500 nm to about 10 μm. In some embodiments, the size of the particles is from about 1 μm to about 7.5 μm. In certain embodiments, the size of the particles is from about 1.5 μm to about 6 μm. In certain embodiments, the size of the particles is from 2 μm to about 3 μm.

In some embodiments, the pharmaceutical composition has a mass median aerodynamic diameter (MMAD) from about 1.0 μm to about 8.0 μm. In some embodiments, the MMAD is from about 1.5 μm to about 6.0 μm. In some embodiments, the MMAD is from about 1.75 μm to about 4.0 μm.

In some embodiments, the pharmaceutical composition has a geometric standard deviation (GSD) from about 1.0 to about 8.0. In some embodiments, the GSD is from about 1.25 to about 6.0. In certain embodiments, the GSD is from about 1.5 to about 4.0. In some embodiments, the pharmaceutical composition has a fine powder fraction of the recovered dose of greater than 30%. In certain embodiments, the fine powder fraction of the recovered dose is greater than 40%. In certain embodiments, the fine powder fraction of the recovered dose is greater than 50%. In certain embodiments, the pharmaceutical composition has an emitted dose of the recovered dose of greater than 70%. In certain embodiments, the emitted dose of the recovered dose is greater than 80%. In certain embodiments, the emitted dose of the recovered dose is greater than 85%.

In some aspects, the present disclosure provides a pharmaceutical composition comprising:
(A) an active pharmaceutical ingredient;
(B) a co-former; and
(C) an excipient;
wherein the active pharmaceutical ingredient and the co-former forms a co-crystal, the co-crystal and the excipient are formulated into a single particle, and the co-crystal of the
active pharmaceutical ingredient and the co-former have particles from about 500 nm to about 15 μm.

In some embodiments, the co-former comprises an acid group selected from either a phosphoric acid group, a sulfuric acid group, or carboxylic acid group. In some embodiments, the co-former comprises a carboxylic acid group. In some embodiments, the co-former comprises one or two carboxylic acid groups, such as an aliphatic carboxylic acid. In some embodiments, the co-former comprises from 1-8 carbon atoms. In some embodiments, the co-former is an aliphatic carboxylic acid having from 3-8 carbon atoms. In certain embodiments, the co-former is an aliphatic carboxylic acid having from 3-6 carbon atoms. In certain embodiments, the co-former is an aliphatic carboxylic acid having 1 carbon atom, such as formic acid. In some embodiments, the co-former is an aliphatic carboxylic acid having 2 carbon atoms, such as acetic acid or trifluoroacetic acid. In some embodiments, the co-former is an aliphatic carboxylic acid having 4 carbon atoms, such as succinic acid or fumaric acid. In certain embodiments, the co-former is succinic acid. In other embodiments, the co-former is fumaric acid. In some embodiments, the co-former is an aliphatic carboxylic acid having six carbon atoms, such as citric acid. In some embodiments, the co-former is a sulfuric acid group or phosphoric acid group. In some embodiments, the inorganic acid is phosphoric acid.

In some embodiments, co-former comprises a basic group, such as an aliphatic group comprising a basic group. In some embodiments, the aliphatic group comprising a basic group has from 1 to 8 carbon atoms. In some embodiments, the aliphatic group comprising a basic group has from 3 to 8 carbon atoms. In some embodiments, the basic group is an amine group, such as a primary amine group, a secondary amine group, a tertiary amine group, or a quarternary amine group.

In some embodiments, the co-former is a second active pharmaceutical ingredient. In some embodiments, the second active pharmaceutical ingredient is different from the active pharmaceutical ingredient. In some embodiments, the second active pharmaceutical ingredient comprises an acidic group, such as a carboxylic acid or mycophenolic acid. In some embodiments, the second active pharmaceutical ingredient comprises a basic group, such as an amine group.

In some embodiments, the pharmaceutical composition comprises a molar ratio of the active pharmaceutical ingredient and co-former from about 1:5 to about 10:1. In some embodiments, the molar ratio is from about 1:2 to about 5:1. In certain embodiments, the molar ratio is from about 1:2 to about 2:1. In certain embodiments, the molar ratio is about 1:1. In certain embodiments, the molar ratio is about 2:1.

In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives. In some embodiments, the active pharmaceutical ingredient is an anti-inflammatory agent, such as an anti-fibrotic agent. In some embodiments, the anti-inflammatory agent is pirfenidone.

In some embodiments, the excipient is a sugar or sugar alcohol. In some embodiments, the excipient is a sugar, such as a polysaccharide. In some embodiments, the excipient is a disaccharide, such as trehalose or lactose. In some embodiments, the excipient is lactose. In some embodiments, the excipient is lactose that has been milled with a particle size. In some embodiments, the particle size has a D₅₀ as measured by laser diffraction from about 10 μm to about 150 μm. In some embodiments, the particle size has a D₅₀ as measured by laser diffraction from about 50 μm to about 125 μm. In some embodiments, the particle size has a D₅₀ as measured by laser diffraction from about 75 μm to about 100 μm.

In some embodiments, the excipient is a sugar alcohol, such as mannitol. In some embodiments, the excipient is an amino acid or a peptide. In some embodiments, the excipient is an amino acid, such as a hydrophobic amino acid. In some embodiments, the excipient is an aliphatic hydrophobic amino acid. In some embodiments, the excipient is leucine. In some embodiments, the excipient is a polar amino acid, such as lysine. In some embodiments, the excipient is glycine.

In some embodiments, the pharmaceutical composition comprises particles of the co-crystal having a size from about 250 nm to about 10 μm. In some embodiments, the size of the particles is from about 1.25 μm to about 7.5 μm. In certain embodiments, the size of the particles is from about 1.5 μm to about 6 μm. In certain embodiments, the size of the particles is from 2 μm to about 3 μm.

In some embodiments, the pharmaceutical composition has a mass median aerodynamic diameter (MMAD) from about 1.0 μm to about 8.0 μm. In certain embodiments, the MMAD is from about 1.5 μm to about 6.0 μm. In certain embodiments, the MMAD is from about 1.75 μm to about 4.0 μm. In some embodiments, the pharmaceutical composition has a geometric standard deviation (GSD) from about 1.0 to about 8.0. In certain embodiments, the GSD is from about 1.25 to about 6.0. In certain embodiments, the GSD is from about 1.5 to about 4.0.

In some embodiments, the pharmaceutical composition has a fine powder fraction of the recovered dose of greater than 30%. In certain embodiments, the fine powder fraction of the recovered dose is greater than 40%. In certain embodiments, the fine powder fraction of the recovered dose is greater than 50%. In certain embodiments, the pharmaceutical composition has an emitted dose of the recovered dose of greater than 70%. In certain embodiments, the emitted dose of the recovered dose is greater than 80%. In certain embodiments, the emitted dose of the recovered dose is greater than 85%.

In some aspects, the present disclosure provides a pharmaceutical composition comprising:
(A) pirfenidone;
(B) a co-former; wherein the co-former is a C1-C8 aliphatic carboxylic acid; and
(C) an excipient, wherein the excipient is mannitol, leucine, or lactose.

In some embodiments, the pharmaceutical composition is formulated for administration via inhalation. In some embodiments, the pharmaceutical composition is formulated for administration via injection, orally, or via inhalation. In some embodiments, the pharmaceutical composition is formulated as a unit dose. In some embodiments, the pharmaceutical composition is formulated for use with an inhaler. In some embodiments, the inhaler is a fixed dose combination inhaler, a single dose dry powder inhaler, a multi-dose dry powder inhaler, multi-unit dose dry powder inhaler, a metered dose inhaler, or a pressurized metered dose inhaler. In some embodiments, the inhaler is a capsule-based inhaler. In some embodiments, the inhaler is a low resistance inhaler. In other embodiments, the inhaler is a high resistance inhaler. In some embodiments, the inhaler is used with a flow rate from about 10 L/min to about 150 L/min. In some embodiments, the flow rate is from about 20 L/min to about 100 L/min. In some embodiments, the inhaler has a loaded dose from about 0.1 mg to about 500 mg. In some embodiments, the inhaler has a loaded dose from about 0.1 mg to about 100 mg. In certain embodiments, the inhaler has a loaded dose from about 50 mg to about 500 mg. In some embodiments, the loaded dose is from about 5 mg to about 25 mg.

In some embodiments, the inhaler is configured to deliver one or a series of doses from one or more unit doses loaded sequentially. In some embodiments, the inhaler is configured to deliver one dose from one unit dose. In some embodiments, the inhaler is configured to deliver a series of doses from one unit dose. In some embodiments, the inhaler is configured to deliver one dose each from a series of capsules loaded sequentially. In some embodiments, the inhaler is configured to deliver a series of doses from a series of capsules loaded sequentially.

In some aspects, the present disclosure provides a method of treating a disease or disorder comprising administering to the patient in need thereof a therapeutically effective amount of the pharmaceutical composition, wherein the active pharmaceutical ingredient is useful to treating the disease or disorder. In some embodiments, the active pharmaceutical ingredient is useful to prevent the disease or disorder.

In some aspects, the present disclosure provides a kit comprising:
(A) a pharmaceutical composition;
(B) a capsule comprising a unit dose of the pharmaceutical composition, a blister pack comprising a unit dose of the pharmaceutical composition, or a metering device that distributes a unit dose of the pharmaceutical composition; and
(C) an aerosolizing device that disperses the unit dose.

In some embodiments, the aerosolizing device is an inhaler. In some aspects, the kit comprises a capsule comprising a unit dose of the pharmaceutical composition. In some aspects, the kit comprises a blister pack comprising a unit dose of the pharmaceutical composition. In some aspects, the kit comprises a metering device that distributes a unit dose of the pharmaceutical composition.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the X-ray Powder Diffraction (XRPD) of co-crystal pirfenidone and FA prepared by the spray drying process.

FIG. 2 shows the X-ray Powder Diffraction and FTIR of co-crystal pirfenidone and FA prepared by TFFD (F34, F29) and spray drying (S1 and S2).

FIG. 3 shows the SEM of spray-dried powders including pirfenidone and FA at different magnifications (1.00 K×, 3.00 K×, 10.00 K×), illustrating the surface morphology of the imaged samples.

FIG. 4 shows SEM of TFFD and spray-dried powders including pirfenidone and FA at different magnifications (1.00 K×, 3.00 K×, 10.00 K×), illustrating the surface morphology of the imaged samples.

FIG. 5 shows the drug deposition of co-crystal pirfenidone prepared by spray drying (S1, S2, S3, S4 and S5).

FIG. 6 shows the drug deposition of cocrystal pirfenidone prepared by TFFD (F23, F29) and spray drying (S1 and S2).

FIG. 7 shows the X-ray Powder Diffraction and FTIR of TFFD formulations using drying cycles 1 and 2.

FIG. 8 shows the SEM of TFFD powders, including pirfenidone and FA, prepared by drying cycles 1 and 2 at a magnification of 10.00 K×, illustrating the surface morphology of the imaged samples.

FIG. 9 shows the Dynamic Vapor Sorption (DVS) isotherm of TFFD cocrystal (F23).

FIG. 10 shows the drug deposition of TFFD co-crystal pirfenidone prepared by drying cycles 1 and 2.

FIG. 11 shows the XRPD and FTIR of cocrystal PIR-MA (F42) compared with cocrystal PIR-FA (F23).

FIG. 12 shows the SEM showing surface morphology of co-crystal pirfenidone and MA at different magnifications (500×, 1.00 K×, 3.00 K×, 5.00 K×, 10.00 K×)

FIG. 13 shows the drug deposition of cocrystal pirfenidone and MA (F42)

### DESCRIPTION OF ILLUSTRATIVE EMBODIMENTS

In some aspects, the present disclosure relates to methods of preparing pharmaceutical compositions comprising one or more composite particles containing an active pharmaceutical ingredient, a co-former, and an excipient or carrier capable of being delivered to the upper and lower airways in the treatment of diseases, where the active pharmaceutical ingredient and the co-former form a co-crystal. The composite particles are engineered in such a way that the resulting composition may be delivered in powder form using a dry powder inhaler (DPI) to the lower airways. The ability to deliver the pharmaceutical compositions using a range of delivery systems without the need for changes to the powder components and ratios or processing methods makes the composition broadly applicable to a range of patient populations and includes those who are ambulatory or in an out-patient setting, patients with reduced lung function or those who may require mechanical ventilation, and pediatric or geriatric who may exhibit reduced inspiratory capacity. Also provided herein are compositions prepared using these methods. Details of these methods are provided in more detail below.

### I. Co-Crystals

In some embodiments, the co-crystals and related pharmaceutical compositions described herein comprise an active pharmaceutical ingredient, one or more co-formers, and an excipient. In some embodiments, the active pharmaceutical ingredient and the co-former form a co-crystal. In some embodiments, the co-crystal and the excipient are formulated into a single particle. The co-formers may be either a non-therapeutic excipient such as a flavoring agent, an acid, or a vitamin or in the alternative, the co-former is a second therapeutic agent for either the same disease or a different disease. These two compounds may be present in the co-crystals in a molar ratio from about 10:1 to about 1:10, from about 5:1 to about 1:5, or from about 2:1 to about 1:2. The molar ratio is from about 10:1, about 8:1, about 6:1, about 4:1, about 3:1, about 2:1, about 1:1, about 1:2, about 1:3, about 1:4, about 1:6, or about 1:8, to about 1:10, or any range derivable therein. In some embodiments, the molar ratio is about 1:1. In other embodiments of the present invention, the molar ratio of the active pharmaceutical ingredient and the co-former is 2:1. The compositions described herein may comprise at least 50%, at least 75%, at least 90%, at least 92.5%, at least 95%, at least 98%, or at least 99% of the active pharmaceutical ingredient and the co-former are present as the co-crystal. The co-crystal may have a melting point of less than 500° C., less than 400° C., less than 300° C., less than 250° C., less than 200° C., or less than 150° C. Further details regarding the components of the co-crystal are provided below.

, In some embodiments, the particle size of the co-crystal of a pharmaceutical composition of the present invention is from about 250 nm to about 15 μm. The present co-crystals may exhibit a mean or average particle size distribution greater than 25 μm, greater than 50 μm, or greater than 60 μm. In some embodiments, the co-crystals may exhibit a mean or average particle size from about 250 nm to about 100 μm, about 500 nm to about 90 μm, about 400 nm to about 80 μm, 100 nm to about 70 μm, 50 nm to about 60 μm, 50 μm to about 50 μm, 50 μm to about 40 μm, 55 μm to about 0 μm, or from about 1 μm to 8 μm. The mean or average particle size of a co-crystal comprises from about 500 nm, about 600 nm, about 700 nm, about 800 nm, about 900 nm, about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 5 μm, about 6 μm, about 7 μm, about 8 μm, about 9 μm, or about 10 μm, or any range derivable therein. The mean or average particle size of the pharmaceutical composition may be determined by mesh analysis using a sonic sifter. The particle size distribution of the dried granules can also be determined by a dry laser diffraction technique or scanning electron microscopy.

### A. Active Pharmaceutical Ingredient

The “active pharmaceutical ingredient” used in the present methods refers to any substance, compound, drug, medicament, or other primary active ingredient that provides a therapeutic or pharmacological effect when administered to a human or animal.

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the intestinal cell layer. According to the BCS, drug substances are classified as follows: Class I-High Permeability, High Solubility; Class II-High Permeability, Low Solubility; Class III-Low Permeability, High Solubility; and Class IV-Low Permeability, Low Solubility.

Suitable active pharmaceutical ingredients may be any biologically active agents or a salt, isomer, ester, ether or other derivative, including prodrug, thereof, which include, but are not limited to, anticancer agents, antifungal agents, psychiatric agents such as analgesics, consciousness level-altering agents such as anesthetic agents or hypnotics, nonsteroidal anti-inflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta agonists, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the active pharmaceutical ingredients may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCL, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In some embodiments, the active pharmaceutical ingredient in these compositions is a non-steroidal anti-inflammatory compound such as ibuprofen, meloxicam, tolmetin, indomethacin, nabumetone, fenoprofen, naproxen, diclofenac, difunisal, mefenamic acid, oxaprozin, ketorolac, sulindac, etodolac, piroxicam, or flurbiprofen.

In other embodiments, the active pharmaceutical ingredient is an antimicrobial agent such as antibiotic or anti-viral agent. Some non-limiting examples of antimicrobial agents include obactericidal antibiotics include penicillin, cephalosporin, polymyxin, rifamycin, lipiarmycin, quinolones, and sulfonamides or bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the antimicrobial agent is a drug which acts against mycobacteria such as cycloserine, capreomycin, ethionamide, rifampicin, rifabutin, rifapentine, and streptomycin. Other antibiotics include arsphenamine, chloramphenicol, fosfomycin, fusidic acid, metronidazole, mupirocin, platensimycin, quinupristin, dalfopristin, thiamphenicol, tigecycline, tinidazole, or trimethoprim. In other embodiments, the antimicrobial agents is an antiviral agent such as abacavir, aciclovir, acyclovir, adefovir, amantadine, amprenavir, ampligen, arbidol, atazanavir, atripla, balavir, boceprevirertet, cidofovir, combivir, dolutegravir, daruavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, interferon type I, type II, and type III, lamivudine, lopinavir, loviride, maraviroc, moroxydine, methisazone, nelfinavir, nevirapine, nexavir, oseltamivir, penciclovir, peramivir, pleconaril, podophyllotoxin, raltegravir, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, traporved, valaciclovir, valganciclovir, vicriviroc, vidarabine, viramidine, zalcitabine, zanamivir, or zidovudine. In some embodiments, the antiviral agents is an anti-retroviral, a fusion inhibitor, an integrase inhibitor, an interferon, a nucleoside analogues, a protease inhibitor, a reverse transcriptase inhibitor, a synergistic enhancer, or a natural product such as tea tree oil.

In other embodiments, the active pharmaceutical ingredient is a chemotherapeutic agent. Some non-limiting examples of chemotherapeutic agents include include alkylating agents such as thiotepa and cyclosphosphamide; alkyl sulfonates such as busulfan, improsulfan and piposulfan; aziridines such as benzodopa, carboquone, meturedopa, and methylamelamines including altretamine, uredopa; ethylenimines and triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ₁^{I} and calicheamicin ω₁^{I}; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-1-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

In particular aspects, the active pharmaceutical ingredient may be an anti-inflammatory anti-fibrotic compound such as pirfenidone. Pirfenidone is the first drug approved to treat idiopathic pulmonary fibrosis (IPF). However, as mentioned earlier, patients require high oral doses of up to 1,602 mg/day to achieve therapeutic level in the lungs. Therefore, high oral doses can cause severe adverse effects such as photosensitivity, agranulocytosis, angioedema and drug-induced liver injury. The active pharmaceutical agent may be any other anti-fibrotic compound, such as nintedanib. Compounds which act on the respiratory system, nonlimiting classes of which include antiasthmatic drugs, decongestants, or antitussives, may be active pharmaceutical ingredients according to the present methods. Examples of compounds which act on the respiratory system include arformoterol, aviptadil, denufosol, dornase alfa, fluticasone, guacetisal, guaifenesin, icenticaftor, lucinactant, poractant alfa, revefenacin, and umeclidinium bromide.

### B. Co-Former

In some aspects, the present disclosure comprises one or more compounds formulated into pharmaceutical compositions as co-former with the active pharmaceutical ingredient to form a co-crystal. The co-former may be an excipient such as pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients that may be used in the co-crystals include vitamins, polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the compositions are substantially, essentially, or entirely free of any other excipient other than the co-former. In other embodiments, the composition comprises one or more excipients. In other embodiments, the co-crystals comprise a co-former which is a second active pharmaceutical ingredient rather than an excipient.

In some embodiments, the co-former comprises at least one acidic functional group. The acidic functional groups may be a protic group or a proton donor, or a Lewis acid. The acidic functional groups may be monoprotic or polyprotic. In some embodiments, the co-former comprises at least one carboxylic acid group. In some embodiments, the co-former comprises one or two carboxylic acid groups. A co-former of the present disclosure may comprise 1-8 carbon atoms or 3-8 carbon atoms. The co-former may comprise 1, 2, 3, 4, 5, 6, 7, or 8 carbon atoms, or any range derivable therein, such as from 3-6 carbon atoms. The carbon atoms of the co-formers of the present disclosure may be linked in a branched or non-branched manner. The co-former may comprise aliphatic carbons or aromatic carbons. Aliphatic co-formers of the present disclosure may be saturated or unsaturated. The present disclosure further contemplates halogenated versions of any of the above described carboxylic acids. Non-limiting examples of carboxylic acid-containing compounds which may be co-formers according to the present disclosure include formic acid, acetic acid, trifluoroacetic acid, fumaric acid, succinic acid, and citric acid.

A co-former according to the present disclosure may comprise an inorganic acid, such as phosphoric acid. Additional non-limiting examples of inorganic acids that may be used in the co-crystals include hydrochloric acid, nitric acid, chlorous acid, nitrous acid, sulfuric acid, boric acid, hydrofluoric acid, hydrobromic acid, perchloric acid, or hydroiodic acid.

In another embodiment, the co-former may be a basic group with one or more amine or other postiviely charged moieties. The amine in the co-former may be a primary, secondary, tertiary, or quaternary nitrogen atom. If the amine group in the basic co-former is secondary, tertiary, or quarternary, the amine group may be substituted with one or more alkyl, alkenyl, aryl, or aralkyl groups. These groups may be the derivatives of a fatty acid or triglycerirde and/or may comprise from 1-12 carbon atoms.

The co-former may interact with the active pharmaceutical ingredient through one or more non-covalent interactions. These non-covalent interactions may include ionic interactions, hydrogen bonding, halogen bonding, van der Waals forces, I-x interactions, or hydrophobic effects. The interactions between the co-formers and active pharmaceutical ingredients may comprise two, three, four, five, or six non-covalent interactions which may be the same or a different type of non-covalent interaction. The co-former may interact with the active pharmaceutical ingredients in such a way that it modifies the properties of the active pharmaceutical ingredient including changing its solubility profile. The co-former itself may be sparingly soluble, sensitive to the environment such as the pH or the temperature.

### C. Further Excipients

In some aspects, the present disclosure comprises one or more excipients formulated into pharmaceutical compositions with the co-crystals such as a pharmaceutically acceptable sugar or sugar alcohol. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Furthermore, these compounds may be used as diluents in order to obtain a dosage that can be readily measured or administered to a patient. Non-limiting examples of excipients include polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient.

In some aspects, the pharmaceutical composition may further comprise one or more inorganic or organic material that may be used to bulk up a composition to obtain an effective amount of the compound. The filler may be an inert inorganic or organic compound such as a salt like a calcium, magnesium, sodium, or potassium salt or a sulfate, chloride, or nitrate salt. Commonly used organic compounds include sugars or sugar derivatives, such as carbohydrates, sugars, and sugar alcohols such as mannitol, lactose, starch, or cellulose. The excipient may comprise any number of saccharide units, therefore a) monosaccharides such as glucose, fructose, and galactose; b) disaccharides such as sucrose or trehalose; or c) polysaccharides such as the aforementioned starch or cellulose are non-limiting examples of excipients according to the present disclosure.

In some aspects, the excipient may be an amino acid. The excipient may be an α-amino acid, a β-amino acid, a γ-amino acid, or a δ-amino acid. An amino acid excipient according to the present disclosure may have a polar side-chain or a non-polar side chain. In some embodiments, an amino acid excipient according to the present invention may be polar, such as lysine. In some embodiments, the excipient may be glycine. An amino acid excipient according to the present disclosure may have a hydrophobic side chain or a hydrophilic side chain. In some embodiments, the side chain is hydrophobic, such as leucine. The side chain of an amino acid excipient according to the present disclosure may be aliphatic or aromatic. The side chain of an amino acid excipient according to the present disclosure may comprise a branched or unbranched carbon chain. Multiple amino acids of any identity linked as a peptide of any length is also contemplated as an excipient according to the present application.

In some aspects, the amount of the excipient in the pharmaceutical composition is from about 0.5% to about 99% w/w, from about 1% to about 95% w/w, from about 10% to about 95% w/w, or from about 20% to about 80% w/w. The amount of the excipient in the pharmaceutical composition comprises from about 0.5%, 1%, 2.5%, 5%, 7.5%, 10%, 15%, 20%, 25%, 30%, 35%, 40%, 45%, 50%, 55%, 60%, 65%, 70%, 75%, 80%, 82.5%, 85%, 87.5%, 90%, 91%, 92%, 93%, 94%, 95%, 96%, 97%, 98%, to about 99% w/w, or any range derivable therein, of the total pharmaceutical composition. In one embodiment, the amount of the excipient in the pharmaceutical composition is at 50% to 90% w/w of the total weight of the pharmaceutical composition.

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active pharmaceutical ingredient in a given solution so that the particles formed contain more than one active pharmaceutical ingredient.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid) s, poly(glycolic acid) s, poly(lactic acid-co-glycolic acid) s and blends, combinations, and copolymers thereof.

As stated, excipients and adjuvants may be used in the pharmaceutical composition to enhance the efficacy and efficiency of the active pharmaceutical ingredient in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include sucrose, trehalose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidones, and tyloxapol.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing agent, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

The present excipients may possess one or more favorable properties or may be improve the favorable properties in the pharmaceutical compositions of the present disclosure, such as exhibiting an improved solubility or enhanced bioavailability. Solubility may be reflected in the ratio of peak solubility of the excipient or pharmaceutical composition to the peak solubility of a reference standard agent under the same conditions. Bioavailability can be indicated by the Cmax or the AUC of the excipient or pharmaceutical composition as determined during in vivo testing, where Cmax is the highest reached blood level concentration of the analyte over time of monitoring and AUC is the area under the plasma-time curve. Enhanced bioavailability can be represented as the ratio of Cmax or the AUC of the excipient or pharmaceutical composition of the present disclosure compared to Cmax or the AUC of the reference standard the excipient or pharmaceutical composition under the same conditions.

In some embodiments, the excipients described herein may be subjected to one or more further processing techniques to obtain final dosage form. These methods may involve grinding or milling the excipient prior to the formation of a co-crystal of the present disclosure. The excipients may be ground or milled to obtain a certain value of any of the characterization techniques described in the section that follows. In some embodiments, the excipient is milled. The excipient may be milled to achieve a particular specific surface area, a particular mass median aerodynamic diameter (MMAD), a particular geometric standard deviation (GSD), a particular fine particle fraction, a particular emitted dose, a particular homogeneity, a particular critical primary pressure, a particular Carr's Index, a particular tapped density, or a particular poured density. In some embodiments, the excipient is milled with a particular particle size. The particle size of a milled excipient may be quantified with a D₅₀ value, which is described in further detail below. The D₅₀ of a milled excipient may, in some embodiments, be from about 10 μm to about 150 μm. In some embodiments, the D₅₀ may be from about 50 μm to about 125 μm or from about 75 μm to about 100 μm. The D₅₀ of milled excipients may be about 125 μm, about 115 μm, about 105 μm, about 95 μm, about 85 μm, about 75 μm, about 65 μm, about 55 μm, or about 50 μm. Scanning electron microscopy or laser diffraction characterization methods may be used to determine the particle size and distribution.

### II. Pharmaceutical Compositions

In some aspects, the present disclosure provides pharmaceutical compositions containing an active pharmaceutical ingredient or a pharmaceutically acceptable salt, ester, derivative, analog, pro-drug, or solvates thereof, a co-former, which may be a second active pharmaceutical ingredient, and an excipient as a co-crystal. The present pharmaceutical compositions may be derived from a starting material such as a filament or powder that exhibits one or more favorable properties such as exhibiting a free-flowing property as an angle of repose, sufficient strength, sufficient stress, bend angle, diameter, viscosity, or Carr's Index. The co-crystal may comprise the active pharmaceutical ingredient and the co-former in a molar ratio from about 1:10 to about 10:1, from about 1:4 to about 4:1, or from about 1:2 to about 2:1. The molar ratio of the active pharmaceutical ingredient and the co-former may be between about 1:2 to about 5:1. The molar ratio of the active pharmaceutical may be about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

In some embodiments, the pharmaceutical composition may be defined by one or more favorable properties such as the specific surface area, mass median aerodynamic diameter (MMAD), the geometric standard deviation (GSD), fine particle fraction, emitted dose, homogeneity, critical primary pressure, Carr's Index, tapped density, or poured density.

The present pharmaceutical compositions prepared according to the methods described herein may have a specific surface area from about 2 m²/g to about 100 m²/g, from about 2.5 m²/g to about 50 m²/g, from about 2.5 m²/g to about 25 m²/g, or from about 2.5 m²/g to about 10 m²/g. The specific surface area of the composition may be from about 2 m²/g, 2.5 m²/g, 3 m²/g, 4 m²/g, 5 m²/g, 6 m²/g, 8 m²/g, 10 m²/g, 12.5 m²/g, 15 m²/g, 20 m²/g, 25 m²/g, 30 m²/g, 40 m²/g, 50 m²/g, 75 m²/g, to about 100 m²/g, or any range derivable therein. The specific surface area may be determined by the single-point Braummer-Emmett-Teller (BET) method using a Monosorb rapid surface area analyzer.

Similarly, the present pharmaceutical compositions may have a MMAD that is from about from about 1.0 μm to about 10.0 μm, from about 1.5 μm to about 6.0 μm, or from about 1.75 μm to about 4.0 μm. The MMAD may be from about 0.5 μm, 1.0 μm, 1.5 μm, 2.0 μm, 2.5 μm, 3.0 μm, 3.5 μm, 4.0 μm, 4.5 μm, 5.0 μm, 6.0 μm, 7.5 μm, 8.0 μm, to about 10.0 μm, or any range derivable therein. The MMAD may be measured using scanning electron microscopy as described in the Examples below.

Additionally, the present pharmaceutical compositions may have a GSD that is from about 1.0 to about 10.0, from about 1.0 to about 8.0, about 1.25 to about 6.0, or from about 1.5 to about 4.0. The GSD may be from about 0.5, 1.0, 1.5, 2.0, 2.5, 3.0, 3.5, 4.0, 4.5, 5.0, 6.0, 7.5, 8.0, to about 10.0, or any range derivable therein. The GSD may be measured using scanning electron microscopy as described in the Examples below.

Similarly, the pharmaceutical composition may have fine powder fraction of the recovered dose that is greater than the fine powder fraction of a composition that is prepared using other means such as conventional powder blending. The present pharmaceutical compositions prepared using the methods described herein may have a fine powder fraction that is greater than 10%, greater than 15%, greater than 20%, greater than 25%, greater than 30%, greater than 35%, 40% greater, greater than 45%, greater than 50%, greater than 55%, greater than 60%, greater than 65%, greater than 70%, greater than 75%, greater than 80%, or greater than 90%. The fine particle fraction (FPF) of the recovered dose may be calculated as the total amount of drug collected with an aerodynamic diameter below 5 μm as a percentage of the total amount of drug collected. Similarly, the instant composition may have an emitted dose that is greater than 50%, greater than 60%, greater than 70%, greater than 80%, greater than 85%, greater than 90%, greater than 95%, or greater than 98%. The emitted fraction (EF) may be calculated as the total amount of drug emitted from the device as a percentage of total amount of drug collected.

In other aspects, the present pharmaceutical compositions may comprise particles of the co-crystals described herein. The particles of the co-crystal may exhibit a mean or average particle size distribution greater than 25 μm, greater than 50 μm, or greater than 60 μm. In some embodiments, the pharmaceutical compositions exhibit a mean or average co-crystal particle size from about 250 nm to about 100 μm, about 500 nm to about 90 μm, about 400 nm to about 80 μm, 100 nm to about 70 μm, 50 nm to about 60 μm, 50 μm to about 50 μm, 50 μm to about 40 μm, 55 μm to about 0 μm, or from about 1 μm to 8 μm. The mean or average co-crystal particle size of the pharmaceutical composition comprises from about 500 nm, about 600 nm, about 700 nm, about 800 nm, about 900 nm, about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 5 μm, about 6 μm, about 7 μm, about 8 μm, about 9 μm, or about 10 μm, or any range derivable therein. The mean or average particle size of the co-crystal particles of the pharmaceutical composition may be determined by mesh analysis using a sonic sifter. The particle size distribution of the dried granules can also be determined by a dry laser diffraction technique or scanning electron microscopy.

Similarly, these compositions may exhibit a particle diameter, D₅₀, wherein 50% of the particles in the composition are larger than this particular particle size. The composition may have a D₅₀ of less than 100 μm, less than 75 μm, less than 60 μm, or less than 50 μm. Additionally, the composition have exhibit a particle diameter, D₉₀, wherein 90% of the particles in the composition are smaller than this particular particle size. The particles may have a D₉₀ wherein the D₉₀ is greater than 25 μm, greater than 40 μm, or greater than 50 μm. Alternatively, the D₉₀ may be less than 100 μm, less than 90 μm, less than 80 μm, or less than 75 μm. In some embodiments, the D₉₀ may be from about 10 μm to about 150 μm, from about 25 μm to about 100 μm, from about 50 μm to about 80 μm. The D₉₀ may be from about 10 μm, 25 μm, 30 μm, 40 μm, 45 μm, 50 μm, 55 μm, 60 μm, 65 μm, 70 μm, 75 μm, 80 μm, 85 μm, 90 μm, 10 μm, 100 μm, 105 μm, 110 μm, 120 μm, to about 125 μm, or any range derivable therein. The D₅₀ of the dried granules can be determined by a dry laser diffraction technique or scanning electron microscopy.

These compositions may exhibit one or more free-flowing properties such as having a flowability as measured by the angle of repose of less than 25. The simplest method for the determination of the angle of repose is the “poured” angle. A funnel with a wide outlet is affixed at a distance of 10 cm above the bench, where a piece of paper is placed directly beneath the funnel. The granules are added while the funnel is closed. The contents flow through and collect on the paper. The diameter of the cone (D) and two opposite sides (l₁+l₂) are measured with rulers. The angle of repose (θ) is calculated from the equation arc cos[D/(l₁+l₂)]. The relationship between flow properties and angle of repose has been established. When the angle of repose is less than 25 degrees, the flow is said to be excellent; on the other hand, if the angle of repose is more than 40 degrees, the flow is considered to be poor. These pharmaceutical compositions may be present as agglomerations and used in either a batch, semi-continuous, continuous manufacturing process. The active pharmaceutical ingredient may act as a binder between the absorbent particles within the pharmaceutical composition.

Furthermore, the present compositions preferably have high degree of homogeneity compared to compositions prepared using other methods such conventional powder blending. The homogeneity may be determined by performing the assay of drug in bulk powder and reported as the percentage of drug to the nominal dose. The relative standard deviation of the homogeneity may be calculated by the standard deviation of the drug percentage divided by the average of drug percentage. In some embodiments, the relative standard deviation of the homogeneity of the pharmaceutical compositions prepared using the present methods is less those prepared using conventional methods.

Furthermore, the pharmaceutical compositions when formulated into an inhaler or other similar device may have a critical primary pressure that is greater than a similar composition prepared by jet milling. The critical primary pressure represents a pressure that overcomes interparticulate forces and disperses powder to primary particles or smaller agglomerates.

### A. Inhalation

In some embodiments, the present disclosure relates to respirable particles must be within a particular aerodynamic size range. In some embodiments, the pharmaceutical composition has a MMAD of from about 1.0 to 10.0 microns, from about or 1.5 to about 8 microns, from about 2.0 to about 6.0 microns, or from about 0.5 microns, 1.0 microns, 1.5 microns, 2.0 microns, 2.5 microns, 3.0 microns, 3.5 microns, 4.0 microns, 4.5 microns, 5.0 microns, 6.0 microns, 8.0 microns, 10.0 microns, to about 15.0 microns, or any range derivable therein. In some embodiments, the present disclosure provides methods for the administration of the inhalable pharmaceutical composition provided herein using a device. Administration may be, but is not limited, to inhalation of pharmaceutical using an inhaler. In some embodiments, an inhaler is a simple passive dry powder inhaler (DPI), such as a Plastiape RS01 monodose DPI. In a conventional dry powder inhaler, dry powder is stored in a capsule or reservoir and is delivered to the lungs by inhalation without the use of propellants.

In some embodiments, an inhaler is a single use, disposable inhaler such as a single-dose DPI, such as a DoseOne™, Spinhaler, Rotohaler®, Aerolizer®, or Handihaler. These dry powder inhalers may be a passive DPI. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RS04 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min. Dry powder inhalers may be classified as low resistance, medium resistance or high resistance. Low-resistance inhalers have a resistance of about 0.06 cmH₂O^{0.5} [L/min]⁻¹, while medium-resistance inhalers have a resistance of about 0.09 cmH₂O^{0.5} [L/min]⁻¹, and high-resistance inhalers have a resistance of about 0.163 cmH₂O^{0.5} L/min|⁻¹ (Altman et al., 2018). The pharmaceutical compositions may be used with any of the above classes of inhalers.

In some embodiments, the DPI may utilize a flow rate of between about 5-200 L/min, about 10 L/min to about 150 L/min, or about 20 L/min to about 100 L/min. The flow rate may be about 20 L/min, about 30 L/min, about 40 L/min, about 50 L/min, about 60 L/min, about 70 L/min, about 80 L/min, about 90 L/min, or about 100 L/min, or any range derivable therein.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination. In some embodiments, the inhalable pharmaceutical composition is delivered as a propellant formulation, such as HFA propellants.

In some embodiments, an inhaler is a nebulizer or a soft-mist inhaler such as those described in PCT Publication No. WO 1991/14468 and WO 1997/12687, which are incorporated herein by reference. A nebulizer is used to deliver medication in the form of an aerosolized mist inhaled into the lungs. The medication formulation be aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. In some embodiments, the single use, disposable nebulizer may be used herein. A nebulizer may utilize a flow rate of between about 3-12 L/min, such as about 6 L/min. In some embodiments, the nebulizer is a dry powder nebulizer.

In some embodiments, the composition may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration four times a day, three times a day, twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, the pharmaceutical composition is administered once per day. In preferred embodiments, the pharmaceutical composition is administered less than once per day, such as every other day, every third day, or once per week. I

In some embodiments, the amount of the pharmaceutical composition of the nebulizer or inhaler may be provided in a unit dosage form, such as in a capsule, blister or a cartridge, wherein the unit dose comprises at least 0.05 mg of the pharmaceutical composition, such as at least 0.075 mg or 0.100 mg of the pharmaceutical composition per dose. In some embodiments, the loaded dose is a unit dose. In some embodiments, the loaded dose is greater than a unit dose. An inhaler or a nebulizer may be configured according to the present methods to deliver a dose of a pharmaceutical composition described herein from one unit dose. In some embodiments, the loaded dose may comprise one or more sequentially loaded unit doses. In some embodiments, the present disclosure provides for delivery of one dose of a pharmaceutical composition described herein from one unit dose of several unit doses that have been loaded into an inhaler or nebulizer, such as in several capsules each containing a unit dose. In some embodiments, the present methods provide for the sequential administration of more than one unit dose from a series of unit doses that have been sequentially loaded into an inhaler or nebulizer, again such as a series of sequentially loaded capsules that each contain a unit dose.

In some embodiments, the loaded dose may be from about 0.1 mg to about 500 mg, or about 0.1 mg to about 100 mg. The loaded dose may be less than 1 mg, about 1 mg, about 5 mg, about 6 mg, about 7 mg, about 8 mg, about 9 mg, about 10 mg, about 11 mg, about 13 mg, about 15 mg, about 17 mg, about 19 mg, about 21 mg, about 23 mg, about 25 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 250 mg, about 300 mg, about 400 mg, about 500 mg, or any range derivable therein.

In particular aspects, the unit dosage form does not comprise the administration or addition of any excipient and is merely used to hold the powder for inhalation (i.e., the capsule, blister, or cartridge is not administered). In some embodiments, the entire amount of the powder load may be administered in a high emitted dose, such as at least 1 mg, preferably at least 10 mg, even more preferably 50 mg. In some embodiments, administration of the powder load results in a high fine particle dose into the deep lung such as greater than 1 mg. In some embodiments, the dose may further comprise using a dose from a reservoir or non-unit dose form and the relevant dose is metered out from the device such as a Turbuhaler. In some embodiments, the inhaler may be configured for the administration of more than one dose from one unit dose.

### III. Manufacturing Methods

In some embodiments, the co-crystals described herein may be prepared though a solvent based method such slow evaporation, slow cooling, vapor diffusion, or liquid-liquid diffusion. Alternatively, the co-crystals may be made through a solvent free method such as extrusion. The preparation of the co-crystals described herein may also comprise preparation of solutions of the components, for example preparation of a) a solution of an active pharmaceutical ingredient comprising an active pharmaceutical ingredient and a solvent; b) a solution of a co-former comprising a co-former and a solvent; or c) a solution of an excipient, comprising an excipient and a solvent, with each component described according to the details given above.

The solvents for said solutions may be independently selected from a variety of solvents. In some embodiments, any number of the solvents for solutions of the co-crystal components may be either an organic solvent or an aqueous solvent. In some embodiments, an organic solvent according to the present methods may comprise a compound with 20 carbon atoms, 18 carbon atoms, 16 carbon atoms, 14 carbon atoms, 12 carbon atoms, 10 carbon atoms, 9 carbon atoms, 8 carbon atoms, 7 carbon atoms, 6 carbon atoms, 5 carbon atoms, 4 carbon atoms, 3 carbon atoms, 2 carbon atoms, or 1 carbon atom. In some embodiments, carbon chains of the solvents of any of the solutions may be saturated, unsaturated, aliphatic, aromatic, linear, or branched. Any of the solvents of the present methods may be protic or aprotic. In some embodiments, the present solvents are independently selected from polar protic solvents. In some embodiments, any number of the solvents may be nonpolar. In some embodiments, any number of the solvents of the presently disclosed methods may be independently selected from water and a substituted alkyl compound, such as a cyanoalkyl compound or an alcohol. In some embodiments, any number of the solvents of the presently disclosed methods may be water, acetonitrile, or t-butanol.

In one aspect, as mentioned above, the preparation of the co-crystals and related pharmaceutical compositions of the present disclosure comprises mixing the aforementioned solution of an active pharmaceutical ingredient with the solution of a co-former to produce a co-crystal solution. In some embodiments, the co-crystal solution is mixed with the aforementioned solution of an excipient to produce a precursor pharmaceutical composition. In some embodiments, any number of the solutions defined herein may be further processed before or after any of the mixing steps. In some embodiments, the processing may comprise sonication or heating of the solution for a time period or to a temperature to facilitate dissolution and mixing of the solutes. In some embodiments, the the co-crystal solution is sonicated for 15 minutes prior to mixing with the excipient solution.

The solid content of the precursor pharmaceutical composition may be from about 0.01% w/v to about 15% w/v or from about 0.05% w/v to about 10% w/v. The present methods provide for precursor pharmaceutical compositions with a solid content of about 0.1% w/v, about 0.15% w/v, about 0.20% w/v, about 0.25% w/v, about 0.3% w/v, about 0.35% w/v, about 0.4% w/v, about 0.45% w/v, about 0.5% w/v, about 0.55% w/v, about 0.6% w/v, about 0.65% w/v, about 0.7% w/v, about 0.75% w/v, about 0.8% w/v, about 0.85% w/v, about 0.9% w/v, about 0.95% w/v, about 1.0% w/v, or any range derivable therein. In some embodiments, the precursor pharmaceutical composition comprises a solid content of 0.5% w/v.

In one aspect, the present methods for preparing the co-crystals disclosed herein comprise depositing the precursor pharmaceutical composition onto a surface. In some embodiments, the surface is metallic, such as stainless steel. The precursor pharmaceutical composition may deposited by any means known to one of skill in the art, such as by pipet or by an automated liquid handling or dispensing system. The amount of precursor pharmaceutical composition deposited on the surface may be well-defined, such as an amount from about 0.25 μL to about 50 μL or an amount from about 0.5 μL to about 40 μL. In some embodiments, the amount of precursor pharmaceutical composition deposited on the surface may be about 1 μL to about 30 μL or about 10 μL to about 20 μL. In some embodiments, the amount of precursor pharmaceutical composition deposited on the surface may be about 10 μL, about 10.5 μL, about 11 μL, about 11.5 μL, about 12 μL, about 12.5 μL, about 13 μL, about 13.5 μL, about 14 μL, about 14.5 μL, about 15 μL, about 15.5 μL, about 16 μL, about 16.5 μL, about 17 μL, about 17.5 μL, about 18 μL, about 18.5 μL, about 19 μL, about 19.5 μL, about 20 μL, or any range derivable therein.

In some embodiments, the present methods further comprise depositing the precursor pharmaceutical composition onto the surface from a height. In some embodiments, the height may be a fixed height. In some embodiments, the precursor pharmaceutical composition may be deposited from a single height or from more than one height. The present methods may involve depositing the precursor pharmaceutical composition onto the surface from a height between about 0.5 cm to about 30 cm or from about 1 cm to about 25 cm. In some embodiments, the height is between about 5 cm and about 20 cm, or between about 7.5 cm and about 15 cm. In some embodiments, the preparation of pharmaceutical compositions, co-crystals and precursors of each as disclosed herein may involve depositing the precursor pharmaceutical composition onto the surface from a height of about 1 cm, about 1.5 cm, about 2 cm, about 2.5 cm, about 3 cm, about 3.5 cm, about 4 cm, about 4.5 cm, about 5 cm, about 5.5 cm, about 6 cm, about 6.5 cm, about 7 cm, about 7.5 cm, about 8 cm, about 8.5 cm, about 9 cm, about 9.5 cm, about 10 cm, about 10.5 cm, about 11 cm, about 11.5 cm, about 12 cm, about 12.5 cm, about 13 cm, about 13.5 cm, about 14 cm, about 14.5 cm, about 15 cm, or any range derivable therein.

In some embodiments, the present methods further comprise depositing the precursor pharmaceutical composition onto the surface wherein the surface has a particular temperature. The particular temperature may be at, above, or below room temperature. The particular temperature may be at, above, or below the melting temperature of a co-crystal of the present disclosure. The particular temperature may be reached or maintained by using any appropriate method known to those of skill in the art, such as by using liquid nitrogen to cool the surface. In some embodiments, the present methods further comprise depositing the precursor pharmaceutical composition onto the surface wherein the temperature of the surface is less than 0° C. In some embodiments, the temperature of the surface is less than −10° C. In some embodiments, the temperature of the surface may be between −10° C. and −200° C. or between −50° C. and −175° C. In some embodiments, the temperature of the surface may be between −100° C. and −150° C. In some embodiments, the temperature of the surface may be about −50° C., about −75° C., about −100° C., about −125° C., about −150° C., about −175° C., or about −200° C., or any range derivable therein. In some embodiments, the temperature of the surface may be about −120° C.

In some embodiments, the present methods may further comprise drying the pharmaceutical composition. Any method of drying known to a person of skill in the art may be used according to the present methods. Non-limiting examples of methods of drying include heating or contact with gas or air of an elevated temperature, spray-drying, lyophilization or freeze-drying, or infrared drying. In some embodiments, the present methods may comprise one or more than one drying cycles. The methods may comprises one, two, three, four, five, six, seven, or eight drying cycles. In some embodiments, there are two drying cycles. Each of the drying cycles may have a distinct temperature and a distinct pressure. The highest temperature of any of the drying cycles is below, at, or above the melting temperature of the co-crystal or any excipients such as a sugar or sugar alcohol. In some embodiments, any number of the drying cycles may comprise lyophilization. The first drying cycle may be carried out at a first temperature from about 0° C. to about −120° C. In some embodiments, the first temperature is from about −10° C. to about −80° C., or from about −20° C. to about −60° C. The first temperature that may be used is from about 0° C., −5° C., −10° C., −15° C., −20° C., −25° C., −30° C., −35° C., −40° C., −45° C., −50° C., −55° C., −60° C., −65° C., −70° C., −75° C., −80° C., −85° C., −90° C., −95° C., −100° C., −105° C., −110° C., −115° C., −120° C., or any range derivable therein. As mentioned above, the first drying cycle may be carried out at a first distinct pressure. In some embodiments, the first pressure is reduced compared to atmospheric pressure. The first drying cycle may be carried out at a first pressure from about 5 mTorr to about 500 mTorr. In some embodiments, the first pressure is from about 10 mTorr to about 500 mTorr, 25 mTorr to about 250 mTorr or from about 50 mTorr to about 150 mTorr. The first pressure that may be used is from about 50 mTorr, about 55 mTorr, about 60 mTorr, about 65 mTorr, about 70 mTorr, about 75 mTorr, about 80 mTorr, about 85 mTorr, about 90 mTorr, about 95 mTorr, about 100 mTorr, about 105 mTorr, about 110 mTorr, about 115 mTorr, about 120 mTorr, about 125 mTorr, about 130 mTorr, about 135 mTorr, about 140 mTorr, about 145 mTorr, about 150 mTorr.

In some embodiments, the methods comprise a second cycle of drying the pharmaceutical composition. The second drying cycle may use the same method or a different method than the first drying cycle to dry the pharmaceutical composition. The second drying cycle may therefore be carried out at a second temperature distinct from the temperature of the first drying cycle. The second temperature, according to the present methods, may be from about −10° C. to about 100° C. In some embodiments, the second temperature is from about 0° C. to about 80° C. In some embodiments, the first temperature is from about 10° C. to about 60° C., or about 20° C. to about 50° C. The second temperature that may be used is from about 10° C., 20° C., 25° C., 30° C., 35° C., 40° C., 45° C., 50° C., 55° C., 60° C., 65° C., 70° C., 75° C., 80° C., 85° C., 90° C., 95° C., 100° C., or any range derivable therein. As mentioned above, the second drying cycle may be carried out at a second distinct pressure. In some embodiments, the second pressure is reduced compared to atmospheric pressure. The first drying cycle may be carried out at a first pressure from about 5 mTorr to about 500 mTorr. In some embodiments, the first pressure is from about 10 mTorr to about 500 mTorr, 25 mTorr to about 250 mTorr or from about 50 mTorr to about 150 mTorr. The first pressure that may be used is from about 50 mTorr, about 55 mTorr, about 60 mTorr, about 65 mTorr, about 70 mTorr, about 75 mTorr, about 80 mTorr, about 85 mTorr, about 90 mTorr, about 95 mTorr, about 100 mTorr, about 105 mTorr, about 110 mTorr, about 115 mTorr, about 120 mTorr, about 125 mTorr, about 130 mTorr, about 135 mTorr, about 140 mTorr, about 145 mTorr, about 150 mTorr.

IV. Processing into Final Dosage Form

In some embodiments, the co-crystals or pharmaceutical compositions described herein may be subjected to one or more further processing techniques to obtain final dosage form. In some embodiments, any of the components of the co-crystals, such as an active pharmaceutical ingredient, a co-former, or an excipient, may be subjected to one or more further processing techniques before the co-crystal is formed. These methods may involve grinding or milling the pharmaceutical composition and then compressing the co-crystals into a dosage form. Following any necessary drying steps, the resulting co-crystal or pharmaceutical composition comprising a co-crystal may be milled or ground to granules or a dry powder. The granules or dry powder may then be compacted. Compacting refers to a process whereby a powder mass comprising the granules or powder is densified under high pressure in order to obtain a compact with low porosity, e.g. a tablet. In some embodiments, the composition is formulated in a manner which is amenable to oral administration. Compression of the powder mass is usually done in a tablet press, more specifically in a steel die between two moving punches. Alternatively, these methods may include the use of additive manufacturing techniques to process these compositions into their final dosage form.

In some aspects, the pharmaceutical compositions described herein are processed in a final dosage form. The granules that are produced by the process may be further processed into a capsule or a tablet. Before formulation into a capsule or tablet, the granule may be further milled before being compressed into the capsule or tablet.

### A. Kits

In some cases, active ingredients of the disclosed methods and compositions are preferably not administered to a patient at the same time or by the same route of administration. Therefore, in some embodiments are kits that, when used by the medical practitioner, can simplify the administration of appropriate amounts of active ingredients to a patient.

A typical kit comprises a single unit dosage form of one or more of the therapeutic agents disclosed, alone or in combination with a single unit dosage form of another agent that may be used in combination with the disclosed compositions. Disclosed kits can further comprise devices that are used to administer the active ingredients. Examples of such devices include, but are not limited to, syringes, drip bags, patches, and inhalers.

Disclosed kits can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. However, in specific embodiments, the disclosed formulations do not contain any alcohols or other co-solvents, oils or proteins.

Administration of the pharmaceutical compositions of the present invention to a patient will follow general protocols for the administration of pharmaceuticals, taking into account the toxicity, if any, of the drug. It is expected that the treatment cycles would be repeated as necessary.

The pharmaceutical compositions of the present invention may be administered by a variety of methods, e.g., orally or by injection (e.g., subcutaneous, intravenous, intraperitoneal, etc.). Depending on the route of administration, the active compounds may be coated by a material to protect the compound from the action of acids and other natural conditions which may inactivate the compound. They may also be administered by continuous perfusion/infusion of a disease or wound site. Specific examples of formulations, including a pharmaceutical compositions that showed improved oral bioavailability, are provided in U.S. Patent Application Publication No. 2009/0048204, which is incorporated herein by reference in its entirety. It will be recognized by those skilled in the art that other methods of manufacture may be used to produce dispersions of the present invention with equivalent properties and utility (see, Repka et al., 2002 and references cited therein). Such alternative methods include but are not limited to solvent evaporation, extrusion, such as hot melt extrusion, and other techniques.

To administer the active compound by other than parenteral administration, it may be necessary to coat the compound with, or co-administer the compound with, a material to prevent its inactivation. For example, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The pharmaceutical compositions may also be administered parenterally, intraperitoneally, intraspinally, or intracerebrally. Dispersions may be prepared in, e.g., glycerol, liquid polyethylene glycols, mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The pharmaceutical compositions can be orally administered, for example, with an inert diluent or an assimilable edible carrier. The therapeutic compound and other ingredients may also be enclosed in a hard or soft shell gelatin capsule, compressed into tablets, or incorporated directly into the subject's or patient's diet. For oral therapeutic administration, the pharmaceutical compositions may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects or patients to be treated; each unit containing a predetermined quantity of therapeutic compound calculated to produce the desired therapeutic effect in association with the required pharmaceutical carrier.

The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The pharmaceutical compositions may also be administered topically to the skin, eye, or mucosa. Alternatively, if local delivery to the lungs is desired the pharmaceutical compositions may be administered by inhalation in a dry-powder or aerosol formulation, as described elsewhere in the present application.

The pharmaceutical compositions may be formulated in a biocompatible matrix for use in a drug-eluting stent.

In some embodiments, the effective dose range for the pharmaceutical compositions can be extrapolated from effective doses determined in animal studies for a variety of different animals. In general a human equivalent dose (HED) in mg/kg can be calculated in accordance with the following formula (see, e.g., Reagan-Shaw et al., FASEB J., 22 (3): 659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the pharmaceutical compositions depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present invention or composition comprising a co-crystal of the present invention administered to a subject or a patient may be determined by physical and physiological factors such as age, sex, body weight, severity of condition, the type of disease being treated, previous or concurrent therapeutic interventions, idiopathy of the subject or the patient and on the route of administration. These factors may be determined by a skilled artisan. The practitioner responsible for administration will typically determine the concentration of active ingredient(s) in a composition and appropriate dose(s) for the individual subject or patient. The dosage may be adjusted by the individual physician in the event of any complication.

In some embodiments, the pharmaceutically effective amount is a daily dose from about 0.1 mg to about 500 mg of the compound. In some variations, the daily dose is from about 1 mg to about 300 mg of the compound. In some variations, the daily dose is from about 10 mg to about 200 mg of the compound. In some variations, the daily dose is about 25 mg of the compound. In other variations, the daily dose is about 75 mg of the compound. In still other variations, the daily dose is about 150 mg of the compound. In further variations, the daily dose is from about 0.1 mg to about 30 mg of the compound. In some variations, the daily dose is from about 0.5 mg to about 20 mg of the compound. In some variations, the daily dose is from about 1 mg to about 15 mg of the compound. In some variations, the daily dose is from about 1 mg to about 10 mg of the compound. In some variations, the daily dose is from about 1 mg to about 5 mg of the compound.

In some embodiments, the pharmaceutically effective amount is a daily dose of 0.01-25 mg of compound per kg of body weight. In some variations, the daily dose is 0.05-20 mg of compound per kg of body weight. In some variations, the daily dose is 0.1-10 mg of compound per kg of body weight. In some variations, the daily dose is 0.1-5 mg of compound per kg of body weight. In some variations, the daily dose is 0.1-2.5 mg of compound per kg of body weight.

In some embodiments, the pharmaceutically effective amount is a daily dose of 0.1-1000 mg of compound per kg of body weight. In some variations, the daily dose is 0.15-20 mg of compound per kg of body weight. In some variations, the daily dose is 0.20-mg of compound per kg of body weight. In some variations, the daily dose is 0.40-3 mg of compound per kg of body weight. In some variations, the daily dose is 0.50-9 mg of compound per kg of body weight. In some variations, the daily dose is 0.60-8 mg of compound per kg of body weight. In some variations, the daily dose is 0.70-7 mg of 10 compound per kg of body weight. In some variations, the daily dose is 0.80-6 mg of compound per kg of body weight. In some variations, the daily dose is 0.90-5 mg of compound per kg of body weight. In some variations, the daily dose is from about 1 mg to about 5 mg of compound per kg of body weight.

An effective amount typically will vary from about 0.001 mg/kg to about 1,000 mg/kg, from about 0.01 mg/kg to about 750 mg/kg, from about 0.1 mg/kg to about 500 mg/kg, from about 0.2 mg/kg to about 250 mg/kg, from about 0.3 mg/kg to about 150 mg/kg, from about 0.3 mg/kg to about 100 mg/kg, from about 0.4 mg/kg to about 75 mg/kg, from about 0.5 mg/kg to about 50 mg/kg, from about 0.6 mg/kg to about 30 mg/kg, from about 0.7 mg/kg to about 25 mg/kg, from about 0.8 mg/kg to about 15 mg/kg, from about 0.9 mg/kg to about 10 mg/kg, from about 1 mg/kg to about 5 mg/kg, from about 100 mg/kg to about 500 mg/kg, from about 1.0 mg/kg to about 250 mg/kg, or from about 10.0 mg/kg to about 150 mg/kg, in one or more dose administrations daily, for one or several days (depending, of course, of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range, for example, of 750 mg to 9,000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day, less than 10 mg/kg/day, or less than 5 mg/kg/day. It may alternatively be in the range of 1 mg/kg/day to 200 mg/kg/day. For example, regarding treatment of patients with Alport syndrome, the unit dosage may be an amount that reduces urine protein concentration by at least 40% as compared to an untreated subject or patient. In another embodiment, the unit dosage is an amount that reduces urine protein concentration to a level that is within +10% of the urine protein level of a healthy subject or patient.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 1 mg/kg/body weight to about 5 mg/kg/body weight, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present invention may comprise, for example, at least about 0.1% of a compound of the present invention. In other embodiments, the compound of the present invention may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects or patients may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between.

Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the subject or patient has eaten or will eat.

### V. Definitions

The use of the word “a” or “an”, when used in conjunction with the term “comprising” in the claims and/or the specification, may mean “one,” but it is also consistent with the meaning of “one or more,” “at least one,” and “one or more than one.” As used herein “another” may mean at least a second or more.

As used herein, the terms “active pharmaceutical ingredient”, “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, to alleviate signs or symptoms of the disease. Desirable effects of treatment include decreasing the rate of disease progression, ameliorating or palliating the disease state, and remission or improved prognosis. Alleviation can occur before signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of the cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active pharmaceutical ingredient in the composition is measured by powder x-ray diffraction.

A “poorly soluble drug” refers to a drug that meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to +10% of the indicated value.

As used herein, the term “substantially free of” or “substantially free” in terms of a specified component, is used herein to mean that none of the specified components has been purposefully formulated into a composition and/or is present only as a contaminant or in trace amounts. Preferably, the total amount of all containments, by-products, and other material may be present in that composition in an amount of less than 2%. In the case of materials that may or may not have been purposefully added, the term “essentially free of” or “essentially free” is used to represent that the composition may contain less than 1% of the specific component. The term “entirely free of” or “entirely free” may contain less than 0.1% of the specific component.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the thermally conductive excipient are adjacent to another atom of the thermally conductive excipient.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C. unless otherwise noted. An elevated temperature is a temperature that is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active pharmaceutical ingredient to a patient in a single administration. Such unit dose formulations that may be used include but are not limited to a single tablet, capsule, or other oral formulations, or a single vial with a syringeable liquid or other injectable formulations. The resulting product can then undergo further downstream processing to create an intermediate product, such as granules, that can then be further formulated into a unit dose such as one prepared for oral delivery as tablets, capsules, three-dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

The term “loaded dose” refers to the amount of a compound, pharmaceutical composition, powder, or substance which has been formulated for administration according to any of the methods or technologies described herein. As a non-limiting example, a capsule-based inhaler containing one capsule with 5 mg of a pharmaceutical composition of the present disclosure would have a loaded dose of 5 mg. An inhaler or nebulizer may have a loaded dose that exceeds a unit dose.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pg. 2).

As used herein, the term “physiological pH” refers to a solution with is at its normal pH in the average human. In most situations, the solution has a pH of approximately 7.4.

As used herein, “inhalation” or “pulmonary inhalation” is used to refer to administration of pharmaceutical preparations by inhalation so that they reach the lungs and in particular embodiments the alveolar regions of the lung. Typically, inhalation is through the mouth, but in alternative embodiments in can entail inhalation through the nose.

As used herein, “dry powder” refers to a fine particulate composition that is not suspended or dissolved in an aqueous liquid.

A “non-complex dry powder inhaler” refers to a device for the delivery of medication to the respiratory tract, in which the medication is delivered as a dry powder in a single-use, single-dose manner. In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least either 50% or 80% of the particles by volume are in the median particle diameter range.

A “Mass Median Aerodynamic Diameter (MMAD)” refers to the aerodynamic diameter (different than the geometric diameter) and is measured by cascade impaction, such as by a Next Generation Impactor (NGI apparatus).

The following section provides further details regarding examples of various embodiments. It should be appreciated by those of skill in the art that the techniques disclosed in the examples that follow represent techniques and/or compositions discovered by the inventor to function well. However, those of skill in the art should, in light of the disclosure, appreciate that many changes can be made in the specific embodiments which are disclosed and still obtain a like or similar result without departing from the spirit and scope of the invention. These examples are illustrations of the methods and systems described herein and are not intended to limit the scope of the invention.

### VI. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1: Aerodynamic Performance of Spray-Dried Co-Crystals and Thin-Film Freeze-Dried Co-Crystals

In the thin-film freeze-drying (TFFD) process, pirfenidone (PIR) was dissolved in acetonitrile to obtain a drug solution. Fumaric acid (FA) was dissolved in water to obtain an acid solution, also described herein as a co-crystal solution. The acid solution was added to the drug solution, producing what is termed in the present disclosure a co-crystal solution, then the co-crystal solution was sonicated for 15 minutes. Lactose (Lac) was dissolved in water and then added to the co-crystal solution to give what is defined herein as a precursor pharmaceutical composition. The final mixture of the sample solution was 50% v/v acetonitrile/water with a solid content of 0.5% w/v (Table 1)

TABLE 1
Compositions formulated for evaluation of drying method

| | | | | | Molar | | |
|---|---|---|---|---|---|---|---|
| | Pir | FA | Lac | Leu | ratio | | Process |
| Formulation | (% w/w) | (% w/w) | (% w/w) | (% w/w) | PIR:acid | Method | Parameters |
| S1 | 76.14 | 23.86 | — | — | 2:1 | Spray | Inlet; 100° C. |
| | | | | | | drying | Feed; 1 ml/min |
| S2 | 38.07 | 23.86 | 19.04 | 19.04 | 1:1 | Spray | Inlet; 100° C. |
| | | | | | | drying | Feed; 1 ml/min |
| S3 | 76.14 | 23.86 | — | — | 2:1 | Spray | Inlet; 100° C. |
| | | | | | | drying | Feed; 3 ml/min |
| S4 | 76.14 | 23.86 | — | — | 2:1 | Spray | Inlet; 80° C. |
| | | | | | | drying | Feed; 1 ml/min |
| S5 | 76.14 | 23.86 | — | — | 2:1 | Spray | Inlet; 80° C. |
| | | | | | | drying | Feed; 3 ml/min |
| F34 | 76.14 | 23.86 | — | — | 2:1 | TFFD | Freezing temp; −120° C. |
| F29 | 38.07 | 23.86 | 19.04 | 19.04 | 1:1 | TFFD | Freezing temp; −120° C. |

Formulations with an S prefix were dried by spray-drying.
Formulations with an F prefix were dried by thin-film freeze-drying.

Approximately 15 μL of each formulated sample solution was dropped from a height of 10 cm onto a stainless-steel rotating drum cooled by liquid nitrogen (LN) that cooled the temperature of the surface to a steady −120° C. The frozen samples were collected into a stainless-steel chamber filled with LN and preserved in a −80° C. freezer before they were transferred to a lyophilizer. The lyophilizer was used to dry frozen samples by removing the solvents. The samples remained at −40° C. for 20 hours, then increased to 25° C. over a period of 20 hours and were maintained at 25° C. for 20 hours for the drying process. Pressure during the drying process was controlled at 100 mTorr.

i. Physicochemical Properties and Co-Crystal Structures

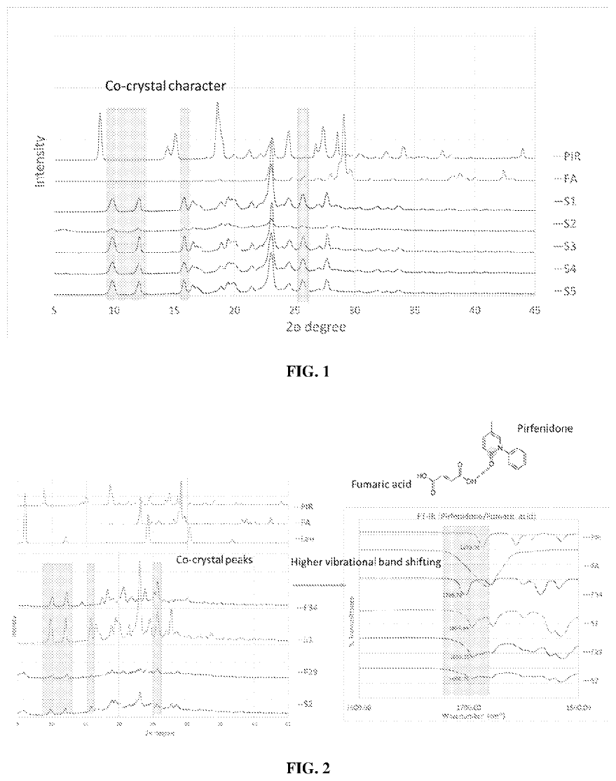

The morphology of TFFD powders was investigated by X-ray Powder Diffraction (XRPD). X-ray diffraction (MiniFlex 600, Rigaku Co., Tokyo, Japan) measured from 5 to 45° over a 20 range (0.02° step, 3°/min, 40 kV, 15 mA). FTIR spectra were evaluated by a Nicolet Fourier transform infrared spectrophotometer (Thermo Fisher Scientific, MA, USA). The spectra were taken with a resolution of 8 cm⁻¹ and were averaged over 128 scans in the 4000-250 cm⁻¹ range of wavelength. The transmittance of each sample was detected. The FTIR spectra, obtained from % transmittance plotted against wavelength (cm⁻¹), presented the intermolecular interaction of pirfenidone and fumaric acid. XRPD of spray-dried formulations prepared with different parameters showed similar co-crystal peaks (FIG. 1).

In comparison, TFFD PIR-FA powders (F23 and F29) and spray-dried powders (S1 and S2) demonstrated the peaks of co-crystal structure. FTIR presented vibrational band shifting of the carbonyl group of pirfenidone at 1673.94 cm⁻¹. TFFD powders (F23) showed higher vibrational band shifting at 1705.76 cm⁻¹, while spray-dried powders (S1) showed vibrational band shifting at 1694.36 cm⁻¹ (FIG. 2). Therefore, without being bound by theory, the TFFD process can create higher intermolecular H-bonding strength than spray drying. However, adding excipients like lactose and leucine, again without being bound by theory, interfered with the intermolecular H-bonding of co-crystal pirfenidone resulting in lower vibrational band shifting. As a result, both TFFD (F29) and spray-dried powders (S2) showed lower intermolecular H-bonding strength compared with TFFD co-crystal powders with no excipient added (F23).

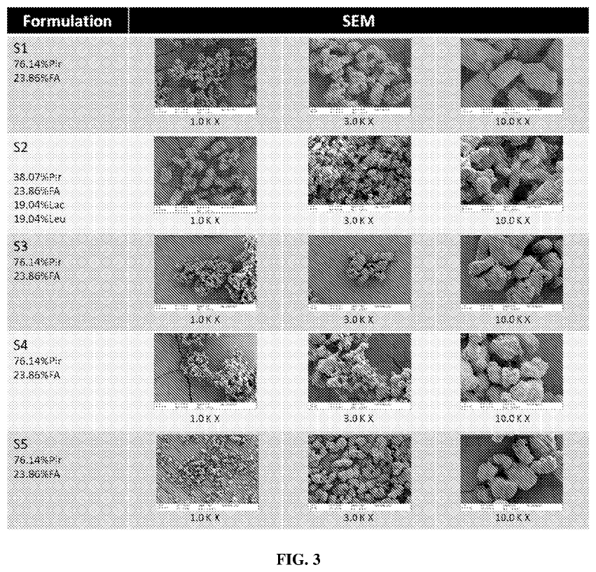

The surface morphology of inhaled powders was investigated by Scanning Electron Microscopy (SEM) (Zeiss Supra 40 V SEM, Carl Zeiss, Heidenheim an der Brenz, Germany). Each sample was coated with Pt/Pd by sputter to reach a thickness of 15 mm. Spray-dried powders showed large particles of pirfenidone over 8 μm. Adjusting process parameters did not change the particle's shape and size, resulting in a similar particle agglomeration (FIG. 3).

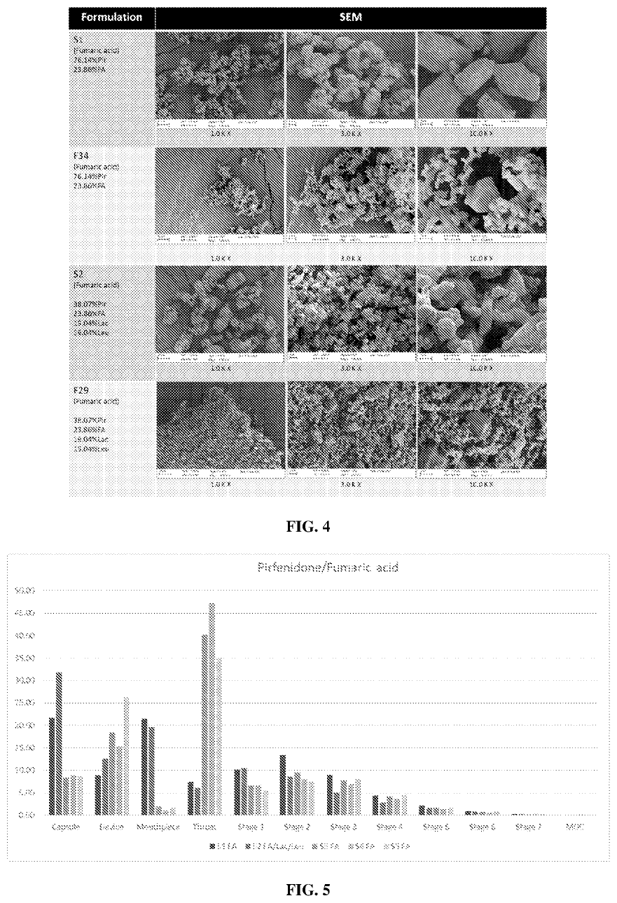

In comparing spray-drying and TFFD powders, co-crystal PIR-FA (F23, F29) prepared by TFFD demonstrated co-crystal pirfenidone particle (~2-3 μm) dispersed in the brittle matrix powder. While co-crystal PIR-FA prepared by spray drying (S1) showed larger particles of co-crystal pirfenidone that agglomerated to large particles over 8 μm. Adding excipients and decreasing drug loading improved the morphology of TFFD powders. F29 exhibited co-crystal particles about 2-3 μm suspended by brittle matrix powders of excipients. However, spray drying produced a mixture of small (4-6 μm) and large particles (over 10 μm) that were aggregated to dense powders (S2) (FIG. 4). Therefore, TFFD not only produced co-crystal pirfenidone but also improved the surface morphology of inhaled pirfenidone powders to enhance aerodynamic performance.

ii. Aerodynamic Particle Size Distribution

Aerodynamic particle size distribution was investigated by a Next Generation Pharmaceutical Impactor (NGI) (MSP Co. Shoreview, MN) connected with a High Capacity Pump (model HCP5, Copley Scientific, Nottingham, UK), and Critical Flow Controller (model TPK 2000, Copley Scientific, Nottingham, UK) was used to control air flow rate. An HPMC capsule no. 3 containing TFFD powders (approximately 5 mg per capsule) was loaded into a high-resistant RS00 dry powder inhaler (Plastiape, Osnago, Italy). TFFD powders (5 mg) were delivered into the NGI through the USP induction port, stages 1-7, and micro-orifice collector (MOC) at the flow rate of 58 L/min for 4 s per each actuation. Acetonitrile and water at 1:1 ratio were extraction solvents to dissolve drug content completely for each stage.

High-Performance Liquid Chromatography (HPLC) carried out with a Dionex Ultimate 3000 HPLC system (Sunnyvale, CA) with a Waters Xbridge C18 column (4.6× 150 mm, 3.5 μm) (Milford, MA) was used to quantify drug contents. A mobile phase A was water adding phosphoric acid to pH 3 and a mobile phase B was 25% v/v of acetonitrile to 7 minutes, 60% v/v of acetonitrile to 8 minutes, and 25% v/v of acetonitrile to 9 minutes. The retention time of pirfenidone was 4.51 minutes.

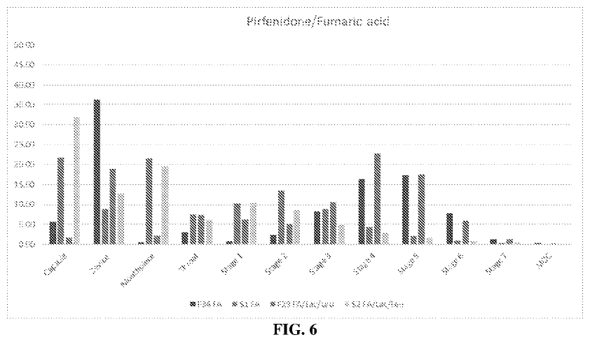

Spray-dried formulations showed low % FPF (recovered) of 11-16% and large MMAD of 4-6 μm. Changing parameters did not significantly enhance aerodynamic performance and drug deposition (Table 2 and FIG. 5) In the comparison, formulations containing 76.14% pirfenidone and 23.86% FA (2:1), S1 showed % FPF (recovered) of 16.32±2.35% and MMAD of 5.31±0.06 μm. However, the TFFD formulation of F34 showed % FPF (recovered) of 57.28±3.92% and MMAD of 1.84±0.15 μm. Formulations containing 38.07% pirfenidone and 23.86% FA (1:1), S2 showed % FPF (recovered) of 11.21±0.73% and MMAD of 5.91±0.13 μm. On the other hand, the TFFD formulation of F29 showed % FPF (recovered) of 53.31±6.69% and MMAD of 2.67±0.23 μm (Table 2). Drug deposition evaluation revealed that F34 and F29 deposited NGI stages 3-7, resulting in higher % FPF and lower MMAD than S1 and S2 (FIG. 6). Based on the aerodynamic performance, the TFFD process produced co-crystal pirfenidone that showed increased aerodynamic performance compared with spray-dried powders.

TABLE 2
Aerodynamic performance of each formulation

| | | | Molar | FPF | FPF | | | |
|---|---|---|---|---|---|---|---|---|
| | | FA | ratio | (%, | (%, | MMAD | | ED |
| Formulation | PIR % w/w | % w/w | (PIR:acid) | recovered) | delivered) | (μm) | GSD | (%) |
| S1 | 76.14 | 23.86 | 2:1 | 16.22 ± 1.07 | 22.19 ± 0.76 | 4.83 ± 0.19 | 2.02 ± 0.25 | 77.64 ± 7.14 |
| S2 | 38.07 | 23.86 | 1:1 | 11.21 ± 0.73 | 16.20 ± 4.81 | 5.91 ± 0.13 | 2.45 ± 0.01 | 72.08 ± 4.44 |
| S3 | 76.14 | 23.86 | 2:1 | 16.32 ± 0.98 | 22.33 ± 0.77 | 4.74 ± 0.18 | 2.04 ± 0.23 | 73.12 ± 4.51 |
| S4 | 76.14 | 23.86 | 2:1 | 14.34 ± 1.15 | 18.87 ± 0.76 | 4.80 ± 0.04 | 2.03 ± 0.03 | 75.89 ± 2.97 |
| S5 | 76.14 | 23.86 | 2:1 | 16.94 ± 0.57 | 26.03 ± 0.31 | 4.23 ± 0.10 | 1.99 ± 0.12 | 65.09 ± 2.87 |
| F34 | 76.14 | 23.86 | 2:1 | 57.28 ± 3.92 | 89.39 ± 1.01 | 1.84 ± 0.15 | 1.88 ± 0.11 | 61.55 ± 1.56 |
| F29 | 38.07 | 23.86 | 1:1 | 53.31 ± 6.69 | 72.2 ± 3.00 | 2.67 ± 0.23 | 2.21 ± 0.04 | 75.15 ± 6.10 |

(n = 3, average ± sd)

### Example 2: Development of Drying Cycles for Co-Crystals Produced by the TFFD Process

i. Pirfenidone Co-Crystal Preparation

Pirfenidone (PIR) was dissolved in acetonitrile to obtain a drug solution. Fumaric acid (FA) was dissolved in water to obtain an acid solution, also known herein as a co-former solution. The acid solution was added to the drug solution to give a solution termed herein as a co-crystal solution, then the co-crystal solution was sonicated for 15 minutes. Lactose (Lac) was well dissolved in water and then added to the co-crystal solution to give what is termed herein as a precursor pharmaceutical composition. The final mixture of the sample solution was 50% v/v acetonitrile/water with solid content, as shown in Table 3. Approximately 15 μL of each sample solution was dropped from 10 cm height onto a stainless-steel rotating drum cooled by liquid nitrogen (LN) to a steady −120° C. The frozen samples were collected into a stainless-steel chamber filled with LN and preserved in a −80° C. freezer before being transferred to a lyophilizer. Each method of the drying cycle (Table 4) was used to dry frozen samples by removing the solvents.

TABLE 3
Formulated Compositions

| | | | | | Solid | | |
|---|---|---|---|---|---|---|---|
| | Pir | FA | Lac | Leu | content | Molar ratio | Drying |
| Formulation | (% w/w) | (% w/w) | (% w/w) | (% w/w) | (% w/w) | PIR:acid | cycle |
| P01_1 | 50.00 | — | 50.00 | — | 1.00 | — | Cycle 1 |
| P01_2 | 50.00 | — | 50.00 | — | 1.00 | — | Cycle 2 |
| F18_1 | 61.48 | 38.52 | — | — | 0.50 | 1:1 | Cycle 1 |
| F18_2 | 61.48 | 38.52 | — | — | 0.50 | 1:1 | Cycle 2 |
| F23_1 | 76.14 | 23.86 | — | — | 0.50 | 2:1 | Cycle 1 |
| F23_2 | 76.14 | 23.86 | — | — | 0.50 | 2:1 | Cycle 2 |
| F29_1 | 38.07 | 23.86 | 19.04 | 19.04 | 0.50 | 1:1 | Cycle 1 |
| F29_2 | 38.07 | 23.86 | 19.04 | 19.04 | 0.50 | 1:1 | Cycle 2 |

TABLE 4
Drying cycle parameters for evaluation of lyophilization procedure

| | | | | | | | | Secondary | |
|---|---|---|---|---|---|---|---|---|---|
| Drying | Shelf | Primary drying | | | | | | drying | Total time |
| cycle | load | Hold | Ramp | Hold | Ramp | Hold | Ramp | Hold | (min) |
| Cycle 1 | −40° C. | −40° C., | — | — | — | — | 25° C., | 25° C., | 3600 |
| | | 1200 min, | | | | | 1200 min, | 1200 min, | |
| | | 100 mTorr | | | | | 100 mTorr | 100 mTorr | |
| Cycle 2 | −50° C. | −50° C., | −40° C., | −40° C., | −20° C., | −20° C., | 25° C., | 25° C., | 2370 |
| | | 60 min, | 120 min, | 360 min, | 240 min, | 240 min, | 900 min, | 450 min, | |
| | | 100 mTorr | 100 mTorr | 100 mTorr | 100 mTorr | 100 mTorr | 100 mTorr | 100 mTorr | |

ii. Co-Crystal Structures

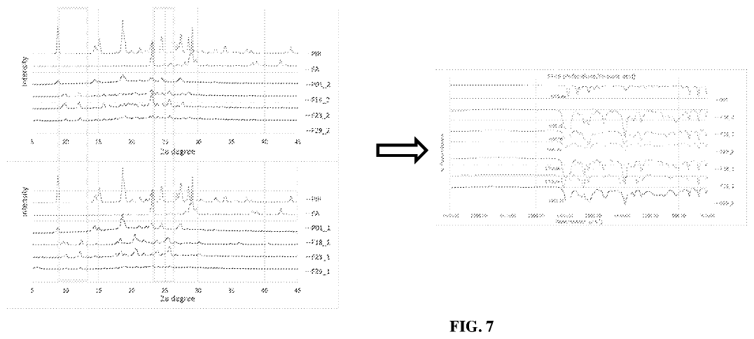

The morphology of TFFD powders was investigated by X-ray Powder Diffraction (XRPD). X-ray diffraction (MiniFlex 600, Rigaku Co., Tokyo, Japan) was measured from 5 to 45° over a 20 range (0.02° step, 3°/min, 40 kV, 15 mA). FTIR spectra were evaluated by a Nicolet Fourier transform infrared spectrophotometer (Thermo Fisher Scientific, MA, USA). The spectra were taken with a resolution of 8 cm⁻¹ and were averaged over 128 scans in the 4000-250 cm⁻¹ range of wavelength. The transmittance of each sample was detected. The FTIR spectra, obtained from % transmittance plotted against wavelength (cm⁻¹), presented the intermolecular interaction of pirfenidone and fumaric acid. XRPD of spray-dried formulations showed co-crystal peaks. TFFD PIR-FA powders prepared by drying cycles 1 and 2 demonstrated similar peaks of co-crystal structure. FTIR presented vibrational band shifting of the carbonyl group of pirfenidone at 1673.94 cm⁻¹. The TFFD powders (F23_1) dried by drying cycle 1 showed slightly higher vibrational band shifting at 1705.76 cm⁻¹, while TFFD powders (F23_2) dried by drying cycle 2 showed a vibrational band shifting at 1696.09 cm⁻¹ (FIG. 7). Without being bound by theory, adding excipients such as lactose and leucine interfered with the intermolecular H-bonding of co-crystal pirfenidone, resulting in lower vibrational band shifting.

iii. Morphology of TFFD Co-Crystals

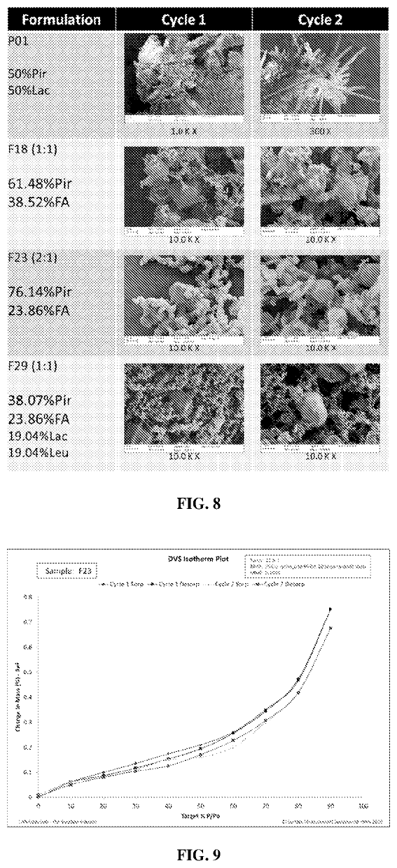

The surface morphology of inhaled powders was investigated by Scanning Electron Microscopy (SEM) (Zeiss Supra 40 V SEM, Carl Zeiss, Heidenheim an der Brenz, Germany). Each sample was coated with Pt/Pd by sputter to reach a thickness of 15 mm. SEM images of TFFD pirfenidone samples that were prepared by drying cycles 1 and 2 showed similar particle shape and size distinct from lactose. Regarding PIR-FA co-crystals, TFFD powders of co-crystal prepared by drying cycles 1 and 2 demonstrated co-crystal pirfenidone particle (~2-3 μm) dispersed in the brittle matrix powder (FIG. 8). Additionally, the TFFD co-crystals prepared by drying cycle 2 showed larger space between particles, resulting in higher porosity of the powders (FIG. 8). Therefore, the TFFD co-crystals prepared by drying cycle 2 showed that larger space between particles might increase specific surface area.

iv. Specific Surface Area (SSA)

Specific surface area (SSA) was evaluated by a Monosorb MS-21 surface area analyzer (Quantachrome, Boynton Beach, FL, USA). Each TFFD formulation was accurately weighed and outgassed at 25° C. by nitrogen purging (20 psi) for at least 18 h to provide a clean powder surface for analysis. The outgassing temperature was selected at 25° C. to avoid heat-related degradation. SSA was calculated by the surface area (m²) and the sample weight (g). The data analysis was performed in triplicate measurements. The average and standard deviation for the analyzed formulations is presented in Table 5. TFFD co-crystals of F18_2, F23_2 and F29_2 that were prepared by drying cycle 2 showed an significantly increased SSA compared with drying cycle 1 (F18_1, F23_1 and F29_1). For example, F18_2 showed SSA of 8.73±0.17 m²/g, while F18_1 showed SSA of 5.63±0.35 m²/g. Therefore, the TFFD co-crystals prepared by drying cycle 2 with higher SSA may enhance aerodynamic performance.

TABLE 5
Specific surface area (SSA)

| Formulation | SSA (m²/g) |
|---|---|
| F18_1 | 5.63 ± 0.35 |
| F18_2 | 8.73 ± 0.17 |
| F23_1 | 4.16 ± 0.29 |
| F23_2 | 7.78 ± 0.65 |
| F29_1 | 38.99 ± 1.26 |
| F29_2 | 48.35 ± 2.01 |

v. Thermogravimetric Analysis

The thermogravimetric analysis evaluated the moisture content, onset of degradation and melting point of TFFD co-crystals using a Thermogravimetric Analyzer (Mettler-Toledo, LLC, Columbus, OH, USA). A 70 μL alumina crucible (model number ME-24123) with a lid covering to minimize static evaporation was filled with TFFD-NIN powder (4-5 mg). Temperature ramp experiments were performed from 30° C. to 400° C. at a 10° C./min rate with a nitrogen purge at 50 mL/min. The percentage of moisture content (% w/w) was ascertained when the sample weight changed to plateau before an initial point of degradation. F18 and F23 prepared by drying cycles 1 and 2 showed moisture content below 0.5% w/w (Table 6). However, adding lactose and leucine increased moisture content to 1.69% (F29_2) and 1.75% (F29_1). The onset of degradation (° C.) of TFFD powders prepared by drying cycle 2 showed higher onset of degradation, about 20-30° C.

TABLE 6
Moisture content and thermograms

| | moisture | onset of | |
|---|---|---|---|
| | content | degradation | melting point |
| Formulation | (% w/w) | (° C.) | (° C.) |
| F18_1 | 0.21 | 219.62 | 102.87 |
| F18_2 | 0.29 | 249.33 | 104.08 |
| F23_1 | 0.49 | 231.96 | 103.94 |
| F23_2 | 0.26 | 259.40 | 105.57 |
| F29_1 | 1.75 | 198.58 | 101.98 |
| F29_2 | 1.69 | 223.06 | 103.36 |

vi. Moisture Sorption of the TFFD Co-Crystal

The moisture sorption was evaluated by a dynamic vapor sorption (DVS-1) microbalance (Surface Measurement Systems, London, UK). The TFFD co-crystal (F23) was filled in an alumina pan (approximately 5 mg). Samples were allowed to dry and equilibrate under nitrogen until a baseline initiated less than 0.002% change in dm/dt. The humidity of each step was altered by 10% after equilibrium, which was determined by a dm/dt<0.0075% within 5 min. The complete sorption-desorption cycle was 0% to 90% RH in steps of 10% RH at 25° C. Compared to the initial dry weight, the percentage change in mass was based on the calculation and plot of moisture sorption-desorption isotherms (FIG. 9). Regarding sorption isotherm, the TFFD co-crystal (F23) showed low moisture sorption of 0.75% w/w at 90% RH.

vii. Aerodynamic Particle Size Distribution

Aerodynamic particle size distribution was investigated by a Next Generation Pharmaceutical Impactor (NGI) (MSP Co. Shoreview, MN) connected with a High Capacity Pump (model HCP5, Copley Scientific, Nottingham, UK), and a Critical Flow Controller (model TPK 2000, Copley Scientific, Nottingham, UK) was used to control air flow rate. An HPMC capsule no. 3 containing TFFD powders (approximately 5 mg per capsule) was loaded into a high-resistant RS00 dry powder inhaler (Plastiape, Osnago, Italy). TFFD powders (5 mg) were delivered into the NGI through the USP induction port, stages 1-7, and micro-orifice collector (MOC) at the flow rate of 58 L/min for 4 s per each actuation. Acetonitrile and water at 1:1 ratio were extraction solvents to dissolve drug content completely for each stage.

High-Performance Liquid Chromatography (HPLC), carried out on a Dionex Ultimate 3000 HPLC system (Sunnyvale, CA) with a Waters Xbridge C18 column (4.6×150 mm, 3.5 μm) (Milford, MA), was used to quantify drug contents. A mobile phase A (water adjusted to pH 3 with phosphoric acid) and a mobile phase B (acetonitrile) was 25% v/v of acetonitrile to 7 minutes, 60% v/v of acetonitrile to 8 minutes, and 25% v/v of acetonitrile to 9 minutes. The retention time of pirfenidone was 4.51 minutes.

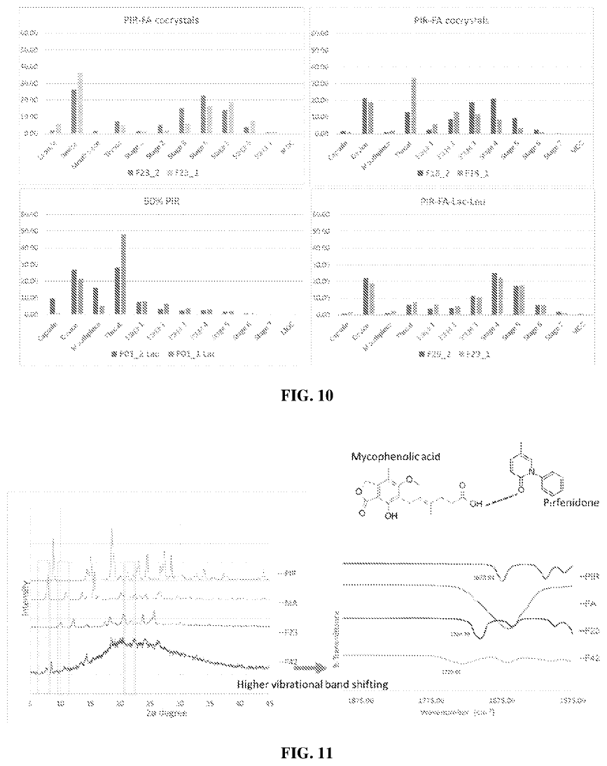

The aerodynamic performance of each formulation prepared by drying cycles 1 and 2 is presented in Table 7. Non-co-crystal formulations of P01 demonstrated similar aerodynamic performance and SSA compared between cycles 1 and 2. However, a suitable drying cycle may modify the physical properties to enhance the aerodynamic performance of TFFD co-crystals. In formulations containing 76.14% pirfenidone and 23.86% FA, F23_1 (cycle 1) showed % FPF of 50.39±1.11% and MMAD of 1.60±0.05 μm, while F23_2 (cycle 2) showed FPF of 57.79±0.69% and smaller MMAD of 2.29±0.10 μm. In terms of 1:1 molar-ratio formulations, F18_1 (cycle 1) showed FPF of 29.71±2.05% and MMAD of 3.97±0.23 μm, while F18_2 (cycle 2) showed an increasing FPF of 54.33±0.51% and decreasing MMAD of 2.77±0.01 μm. F29_1 (cycle 1) showed FPF (recovered) of 53.31±6.69% and MMAD of 2.67±0.23 μm, while F29_2 (cycle 2) showed FPF of 62.76±6.78% and smaller MMAD of 2.04±0.01 μm. Drug deposition evaluation revealed that F18_2 and F23_2 showed increasing drug deposition at NGI stages 3-4 and decreasing at the device and throat (FIG. 10). According to aerodynamic performance, the TFFD process with suitable drying cycles produced co-crystal pirfenidone that showed acceptable aerodynamic performance.

TABLE 7
Aerodynamic performance and SSA of TFFD formulations

| | % FPF | | % FPF | | MMAD | | SSA | |
|---|---|---|---|---|---|---|---|---|
| | (recovered dose) | | (delivered dose) | | (μm) | | (m²/g) | |
| Formulation | Cycle 1 | Cycle 2 | Cycle 1 | Cycle 2 | Cycle 1 | Cycle 2 | Cycle 1 | Cycle 2 |
| P01 | 11.36 ± | 8.66 ± | 14.32 ± | 13.66 ± | 5.46 ± | 5.8 ± | 1.32 ± | 1.62 ± |
| | 0.99 | 0.77 | 1.48 | 0.69 | 0.51 | 0.55 | 0.67 | 0.48 |
| F18 | 29.71 ± | 54.33 ± | 45.63 ± | 70.47 ± | 3.97 ± | 2.77 ± | 5.63 ± | 8.73 ± |
| | 2.05 | 0.51 | 3.57 | 0.71 | 0.23 | 0.01 | 0.35 | 0.17 |
| F23 | 50.39 ± | 57.79 ± | 86.9 ± | 80.39 ± | 1.60 ± | 2.29 ± | 4.16 ± | 7.78 ± |
| | 1.11 | 0.69 | 3.88 | 0.64 | 0.05 | 0.10 | 0.29 | 0.65 |
| F29 | 53.31 ± | 62.76 ± | 72.2 ± | 81.33 ± | 2.67 ± | 2.04 ± | 38.99 ± | 48.35 ± |
| | 6.69 | 6.78 | 3.00 | 1.42 | 0.23 | 0.01 | 1.26 | 2.01 |

### Experiment 3: Pirfenidone and Mycophenolic Acid Co-Crystals

i. Materials

Pirfenidone, fumaric acid (FA), mycophenolic acid (MA) and lactose

ii. Cocrystal Pirfenidone Preparations

In TFFD process, pirfenidone (PIR) was dissolved in acetonitrile to obtain a drug solution. mycophenolic acid (MA) or fumaric acid (FA) was dissolved in water to obtain acid solution. The acid solution was added to the drug solution, then sonicate 15 minutes. Lactose (Lac) was well dissolved in water and then added to the drug-acid solution. The final mixture of the sample solution was 50% v/v acetonitrile/water with a solid content of 0.5% w/v (Table 8). Approximately 15 μL of each sample solution was dropped at 10 cm height onto a stainless-steel rotating drum cooled by Liquid nitrogen (LN) that controlled the temperature of the TFFD process at −120° C. The frozen samples were collected into a stainless-steel chamber filled with LN and preserved in a −80° C. freezer before transferring to a lyophilizer. The lyophilizer was used to dry frozen samples by removing the solvents. The samples were remained at −40° C. for 20 hours, then ramped to 25° C. in 20 hours and sustained at 25° C. for 20 hours for the drying process. Pressure during the drying process was controlled at 100 mTorr.

TABLE 8
Composition of the formulation prepared by TFF process

| | | | | | Molar | |
|---|---|---|---|---|---|---|
| | PIR | FA | MA | Lac | ratio | Solid content |
| Formulation | (% w/w) | (% w/w) | (% w/w) | (% w/w) | PIR:acid | (% w/v) |
| P13 | 25.00 | — | — | 75.00 | — | 0.5 |
| F23 | 76.14 | 23.86 | — | — | 2:1 | 0.5 |
| F42 | 25.00 | — | 21.62 | 53.38 | 1:1 | 0.5 |

iii. Physicochemical Properties and Cocrystals structures

The morphology of TFFD powders was investigated by X-ray Powder Diffraction (XRPD). X-ray diffraction (MiniFlex 600, Rigaku Co., Tokyo, Japan) measured from 5 to 45° over a 20 range (0.02° step, 3°/min, 40 kV, 15 mA). The peaks of the cocrystal were shown in the XRPD pattern. FTIR spectra were evaluated by a Nicolet Fourier transform infrared spectrophotometer (Thermo Fisher Scientific, MA, USA). The spectra were taken with a resolution of 8 cm⁻¹ and were averaged over 128 scans in the 4000-250 cm⁻¹ range of wavelength. The transmittance of each sample was detected. The FTIR spectra, obtained from % transmittance plotted against wavelength (cm⁻¹), presented the intermolecular interaction of pirfenidone and mycophenolic acid (FIG. 30).

In comparison, TFFD PIR-FA powders (F23) and PIR-MA (F42) demonstrated the peaks of cocrystal structure (FIG. 11). FTIR presented vibrational band shifting of the carbonyl group of pirfenidone at 1673.94 cm⁻¹. F42 showed a vibrational band shifting at 1729.86 cm⁻¹, while F23 showed a vibrational band shifting at 1705.76 cm⁻¹ (FIG. 11). Therefore, the TFFD process can create intermolecular H-bonding between pirfenidone and MA.

iv. Morphology of PIR-MA Cocrystals

The surface morphology of inhaled powders was investigated by Scanning Electron Microscopy (SEM) (Zeiss Supra 40 V SEM, Carl Zeiss, Heidenheim an der Brenz, Germany). Each sample was coated with Pt/Pd by sputter to reach a thickness of 15 mm.

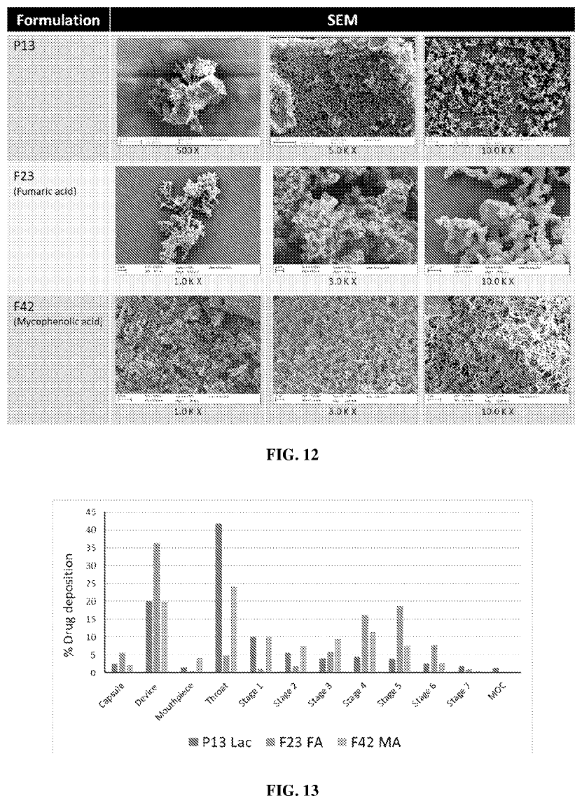

In the non-cocrystal formulation, P13 showed the large rod particle of pirfenidone (over 100 μm) mixed with the brittle matrix powders. In terms of cocrystal formulations, the cocrystal PIR-FA (F23) prepared by TFFD demonstrated cocrystal pirfenidone particle (~2-3 μm) dispersed in the brittle matrix powder. While cocrystal PIR-MA prepared (F42) showed smaller particles of cocrystal pirfenidone lower than 2 μm dispersed in the brittle matrix powder (FIG. 12). However, the inter-particle distance of F23 was larger than F42. Therefore, the TFFD process also produced cocrystal pirfenidone with MA resulting in the changing of particle morphology to enhance aerodynamic performance.

v. Aerodynamic Particle Size Distribution

Aerodynamic particle size distribution was investigated by a Next Generation Pharmaceutical Impactor (NGI) (MSP Co. Shoreview, MN) connected with a High Capacity Pump (model HCP5, Copley Scientific, Nottingham, UK), and Critical Flow Controller (model TPK 2000, Copley Scientific, Nottingham, UK) was used to control air flow rate. An HPMC capsule no. 3 containing TFFD powders (approximately 5 mg per capsule) was loaded into a high-resistant RS00 dry powder inhaler (Plastiape, Osnago, Italy). TFFD powders (5 mg) were delivered into the NGI through the USP induction port, stages 1-7, and micro-orifice collector (MOC) at the flow rate of 58 L/min for 4 s per each actuation. Acetonitrile and water at 1:1 ratio were extraction solvents to dissolve drug content completely for each stage.

High-Performance Liquid Chromatography (HPLC), which was a Dionex Ultimate 3000 HPLC system (Sunnyvale, CA) with a Waters Xbridge C18 column (4.6×150 mm, 3.5 μm) (Milford, MA), was used to quantify drug contents. A mobile phase A was water adding phosphoric acid to pH 3 and a mobile phase B was 25% v/v of acetonitrile to 7 minutes, 60% v/v of acetonitrile to 8 minutes, and 25% v/v of acetonitrile to 9 minutes. The retention time of pirfenidone was 4.51 minutes.

P13 containing pirfenidone and lactose showed % FPF (recovered) of 19.93+6.92 and MMAD of 3.87+1.08 μm. While F42 containing pirfenidone, MA and lactose showed higher % FPF (recovered) of 33.39+4.35 and lower MMAD of 3.17+1.07 μm. However, F23 containing pirfenidone and FA showed the highest % FPF (recovered) of 50.39+1.11 and MMAD of 1.60+0.05 μm (Table 9). Drug deposition evaluation revealed that F23 and F42 deposited NGI stages 3-6, resulting in higher % FPF and lower MMAD than P13 (FIG. 13). Based on the aerodynamic performance, the TFFD process produced cocrystal pirfenidone that showed increasing aerodynamic performance compared with the non-crocrystal formulation.

TABLE 9
Aerodynamic performance of each formulation (n = 3, average ± sd)

| | FPF | FPF | | | |
|---|---|---|---|---|---|
| | (%, | (%, | MMAD | | ED |
| Formulation | recovered) | delivered) | (μm) | GSD | (%) |
| P13 | 19.93 ± 6.92 | 25.37 ± 6.81 | 3.87 ± 1.08 | 4.46 ± 0.58 | 77.70 ± 6.41 |
| F23 | 50.39 ± 1.11 | 86.90 ± 3.88 | 1.60 ± 0.05 | 1.81 ± 0.02 | 57.65 ± 1.56 |
| F42 | 33.39 ± 4.35 | 42.84 ± 0.58 | 3.17 ± 1.07 | 2.28 ± 0.99 | 77.93 ± 4.35 |

TFFD process produced cocrystal pirfenidone with mycophenolic acid that changed the particle morphology of pirfenidone. As a result, the cocrystal pirfenidone and mycophenolic acid improved aerodynamic performance compared with the non-cocrystal formulation.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method of providing a pharmaceutical composition comprising:
(A) admixing a drug solution comprising an active pharmaceutical ingredient and a co-former to form a co-crystal solution;
(B) admixing the co-crystal solution with an excipient to form a precursor pharmaceutical composition;
(C) depositing the precursor pharmaceutical composition onto a surface, wherein the surface is at a surface temperature below the freezing point of the precursor pharmaceutical composition to produce composite drug particles; and
(D) collecting the composite drug particles from the surface to thereby provide the pharmaceutical composition.

2. The method of claim 1, wherein the co-former is added a co-former solution comprising the co-former and a co-former solution solvent.

3. The method of either claim 1 or claim 2, wherein the co-former comprises an acid group selected from either a phosphoric acid group, a sulfuric acid group, or carboxylic acid group.

4. The method according to any one of claims 1-3, wherein the co-former comprises a carboxylic acid group.

5. The method according to any one of claims 1-4, wherein the co-former comprises one or two carboxylic acid groups.

6. The method according to any one of claims 1-5, wherein the co-former is an aliphatic carboxylic acid.

7. The method according to any one of claims 1-6, wherein the co-former is an aliphatic carboxylic acid having from 1-8 carbon atoms.

8. The method according to any one of claims 1-7, wherein the co-former is an aliphatic carboxylic acid having from 3-8 carbon atoms.

9. The method according to any one of claims 1-8, wherein the co-former is an aliphatic carboxylic acid having from 3-6 carbon atoms.

10. The method according to any one of claims 1-8, wherein the co-former is an aliphatic carboxylic acid having 1 carbon atom.

11. The method of claim 10, wherein the co-former is formic acid.

12. The method according to any one of claims 1-8, wherein the co-former is an aliphatic carboxylic acid having 2 carbon atoms.

13. The method of claim 12, wherein the co-former is acetic acid or trifluoroacetic acid.

14. The method according to any one of claims 1-9, wherein the co-former is an aliphatic carboxylic acid having 4 carbon atoms.

15. The method of claim 14, wherein the co-former is succinic acid or fumaric acid.

16. The method of claim 15, wherein the co-former is succinic acid.

17. The method of claim 15, wherein the co-former is fumaric acid.

18. The method according to any one of claims 1-9, wherein the co-former is an aliphatic carboxylic acid having six carbon atoms.

19. The method of claim 18, wherein the co-former is citric acid.

20. The method according to any one of claims 1-3, wherein the co-former is a sulfuric acid group or phosphoric acid group.

21. The method of claim 20, wherein the inorganic acid is phosphoric acid.

22. The method of either claim 1 or claim 2, wherein the co-former comprises a basic group.

23. The method according to any one of claim 1, 2, or 22, wherein the co-former is an aliphatic group comprising a basic group.

24. The method of claim 23, wherein the aliphatic group comprising a basic group has from 1 to 8 carbon atoms.

25. The method of either claim 23 or claim 24, wherein the aliphatic group comprising a basic group has from 3 to 8 carbon atoms.

26. The method according to any one of claims 22-25, wherein the basic group is an amine group.

27. The method according to any one of claims 22-26, wherein the basic group is a primary amine group.

28. The method according to any one of claims 22-26, wherein the basic group is a secondary amine group.

29. The method according to any one of claims 22-26, wherein the basic group is a tertiary amine group.

30. The method according to any one of claims 22-26, wherein the basic group is a quarternary amine group.

31. The method of either claim 1 or claim 2, wherein the co-former is a second active pharmaceutical ingredient.

32. The method of claim 31, wherein the second active pharmaceutical ingredient is different from the active pharmaceutical ingredient.

33. The method of either claim 31 or claim 32, wherein the second active pharmaceutical ingredient comprises an acidic group.

34. The method according to any one of claims 31-33, wherein the second active pharmaceutical ingredient comprises a carboxylic acid.

35. The method according to any one of claims 31-33, wherein the second active pharmaceutical ingredient is mycophenolic acid.

36. The method of either claim 31 or claim 32, wherein the second active pharmaceutical ingredient comprises a basic group.

37. The method according to any one of claims 31, 32, and 36, wherein the second active pharmaceutical ingredient comprises an amine group.

38. The method according to any one of claims 1-37, wherein the method comprises using a molar ratio of the active pharmaceutical ingredient and co-former from about 1:5 to about 10:1.

39. The method of claim 38, wherein the molar ratio is from about 1:2 to about 5:1.

40. The method of either claim 38 or claim 39, wherein the molar ratio is from about 1:2 to about 2:1.

41. The method of claim 40, wherein the molar ratio is about 1:1.

42. The method of claim 40, wherein the molar ratio is about 2:1.

43. The method according to any one of claims 2-42, wherein the co-former solution solvent is a protic solvent.

44. The method of claim 43, wherein the protic solvent is water.

45. The method according to any one of claims 1-44, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

46. The method according to any one of claims 1-45, wherein the active pharmaceutical ingredient is an anti-inflammatory agent.

47. The method of claim 46, wherein the anti-inflammatory agent is an anti-fibrotic agent.

48. The method of either claim 46 or claim 47, wherein the anti-inflammatory agent is pirfenidone.

49. The method according to any one of claims 1-48, wherein the drug solution comprises an active pharmaceutical ingredient and a drug solution solvent.

50. The method of claim 49, wherein the drug solution solvent is an organic solvent.

51. The method of either claim 49 or claim 50, wherein the drug solution solvent is a polar aprotic solvent.

52. The method according to any one of claims 49-51, wherein the drug solution solvent is a C1-C8 cyanoalkyl.

53. The method according to any one of claims 49-52, wherein the drug solution solvent is acetonitrile.

54. The method according to any one of claims 1-53, wherein the excipient is added as an excipient solution comprising an excipient and an excipient solution solvent.

55. The method of claim 54, wherein the excipient is a sugar or sugar alcohol.

56. The method of either claim 54 or claim 55, wherein the excipient is a sugar.

57. The method according to any one of claims 54-56, wherein the excipient is a polysaccharide.

58. The method according to any one of claims 54-57, wherein the excipient is a disaccharide.

59. The method according to any one of claims 54-58, wherein the excipient is trehalose or lactose.

60. The method according to any one of claims 54-59, wherein the excipient is lactose.

61. The method according to any one of claims 54-60, wherein the excipient is lactose that has been milled with a particle size.

62. The method of claim 61, wherein the particle size has a D₅₀ as measured by laser diffraction from about 10 μm to about 150 μm.

63. The method of either claim 61 or claim 62, wherein the particle size has a D₅₀ as measured by laser diffraction from about 50 μm to about 125 μm.

64. The method according to any one of claims 61-63, wherein the particle size has a D₅₀ as measured by laser diffraction from about 75 μm to about 100 μm.

65. The method of claim 55, wherein the excipient is a sugar alcohol.

66. The method of claim 65, wherein the excipient is mannitol.

67. The method of claim 54, wherein the excipient is an amino acid or a peptide.

68. The method of either claim 54 or claim 67, wherein the excipient is an amino acid.

69. The method according to any one of claim 54, 67, or 68, wherein the excipient is a hydrophobic amino acid.

70. The method according to any one of claim 54 or 67-69, wherein the excipient is an aliphatic hydrophobic amino acid.

71. The method according to any one of claim 54 or 67-70, wherein the excipient is leucine.

72. The method according to any one of claim 54, 67, or 68, wherein the excipient is a polar amino acid.

73. The method according to any one of claim 54, 67, 68, or 72, wherein the excipient is lysine.

74. The method according to any one of claim 54, 67, or 68, wherein the excipient is glycine.

75. The method according to any one of claims 54-71, wherein the excipient solution solvent is a protic solvent.

76. The method of claim 75, wherein the excipient solution solvent is water.

77. The method according to any one of claims 54-75, wherein the excipient solution solvent is an organic solvent.

78. The method according to any one of claims 54-77, wherein the excipient solution solvent is a polar protic solvent.

79. The method according to any one of claims 54-78, wherein the solvent is a C1-C8 alcohol.

80. The method according to any one of claims 54-79, wherein the solvent is t-butanol.

81. The method according to any one of claims 1-80, wherein the precursor pharmaceutical composition comprises a solid content from about 0.05% w/v to about 10% w/v.

82. The method of claim 81, wherein the solid content is from about 0.1% w/v to about 5% w/v.

83. The method of claim 82, wherein the solid content is from about 0.25% w/v to about 2.5% w/v.

84. The method of claim 83, wherein the solid content is from about 0.25% w/v to about 1% w/v.

85. The method according to any one of claims 81-84, wherein the solid content is about 0.5% w/v.

86. The method according to any one of claims 1-85, wherein the method further comprises depositing a discrete amount of the precursor pharmaceutical composition onto the surface.

87. The method of claim 86, wherein the discrete amount is from about 0.5 μL to about 40 μL.

88. The method of either claim 86 or claim 87, wherein the discrete amount is from about 1 μL to about 30 μL.

89. The method according to any one of claims 86-88, wherein the discrete amount is from about 10 μL to about 20 μL.

90. The method according to any one of claims 86-89, wherein the discrete amount is 15 μL.

91. The method according to any one of claims 1-90, wherein the method further comprises depositing the precursor pharmaceutical composition onto the surface from a fixed height.

92. The method of claim 91, wherein the fixed height is from about 1 cm to about 25 cm.

93. The method of either claim 91 or claim 92, wherein the fixed height is from about 5 cm to about 20 cm.

94. The method according to any one of claims 91-93, wherein the fixed height is from about 7.5 cm to about 15 cm.

95. The method according to any one of claims 91-94, wherein the fixed height is from about 7.5 cm to about 12.5 cm.

96. The method according to any one of claims 91-95, wherein the fixed heigh is about 10 cm.

97. The method according to any one of claims 1-96, wherein the surface temperature is less than −10° C.

98. The method according to any one of claims 1-97, wherein the surface temperature is from about −10° C. to about −200° C.

99. The method according to any one of claims 1-98, wherein the surface temperature is from about −50° C. to about −175° C.

100. The method according to any one of claims 1-99, wherein the surface temperature is from about −100° C. to about −150° C.

101. The method according to any one of claims 1-100, wherein the surface temperature is about −120° C.

102. The method according to any one of claims 1-101, wherein the method further comprises drying the pharmaceutical composition.

103. The method according to any one of claims 1-102, wherein the drying process comprises lyophilization.

104. The method of claim 103, wherein the drying process comprises two drying cycles.

105. The method of claim 104, wherein the first drying cycle comprises drying at a first temperature from about 0° C. to about −120° C.

106. The method of claim 105, wherein the first temperature is a temperature from about −10° C. to about −80° C.

107. The method of claim 106, wherein the first temperature is a temperature from about −20° C. to about −60° C.

108. The method according to any one of claims 104-107, wherein the first drying cycle comprises drying at a reduced pressure.

109. The method of claim 108, wherein the reduced pressure is a first pressure from about 10 mTorr to about 500 mTorr.

110. The method of claim 109, wherein the first pressure is from about 25 mTorr to about 250 mTorr.

111. The method of claim 110, wherein the first pressure is from about 50 mTorr to about 150 mTorr.

112. The method according to any one of claims 104-111, wherein the second drying cycle comprises drying at a second temperature from about 0° C. to about 80° C.

113. The method of claim 112, wherein the second temperature is a temperature from about 10° C. to about 60° C.

114. The method of claim 113, wherein the second temperature is a temperature from about 20° C. to about 50° C.

115. The method according to any one of claims 104-114, wherein the second drying cycle comprises drying at a reduced pressure.

116. The method of claim 115, wherein the reduced pressure is a second pressure from about 10 mTorr to about 500 mTorr.

117. The method of claim 116, wherein the second pressure is from about 25 mTorr to about 250 mTorr.

118. The method of claim 117, wherein the second pressure is from about 50 mTorr to about 150 mTorr.

119. The method according to any one of claims 1-118, wherein the pharmaceutical composition comprises particles of the co-crystal having a size from about 500 nm to about 10 μm.

120. The method of claim 119, wherein the size of the particles is from about 1 μm to about 7.5 μm.

121. The method of either claim 119 or claim 120, wherein the size of the particles is from about 1.5 μm to about 6 μm.

122. The method according to any one of claims 119-121, wherein the size of the particles is from 2 μm to about 3 μm.

123. The method according to any one of claims 1-122, wherein the pharmaceutical composition has a mass median aerodynamic diameter (MMAD) from about 1.0 μm to about 8.0 μm.

124. The method of claim 123, wherein the MMAD is from about 1.5 μm to about 6.0 μm.

125. The method of either claim 123 or claim 124, wherein the MMAD is from about 1.75 μm to about 4.0 μm.

126. The method according to any one of claims 1-125, wherein the pharmaceutical composition has a geometric standard deviation (GSD) from about 1.0 to about 8.0.

127. The method of claim 126, wherein the GSD is from about 1.25 to about 6.0.

128. The method of either claim 126 or claim 127, wherein the GSD is from about 1.5 to about 4.0.

129. The method according to any one of claims 1-128, wherein the pharmaceutical composition has a fine powder fraction of the recovered dose of greater than 30%.

130. The method of claim 129, wherein the fine powder fraction of the recovered dose is greater than 40%.

131. The method of either claim 129 or claim 130, wherein the fine powder fraction of the recovered dose is greater than 50%.

132. The method according to any one of claims 1-131, wherein the pharmaceutical composition has an emitted dose of the recovered dose of greater than 70%.

133. The method of claim 132, wherein the emitted dose of the recovered dose is greater than 80%.

134. The method of either claim 132 or claim 133, wherein the emitted dose of the recovered dose is greater than 85%.

135. A pharmaceutical composition prepared using a method according to any one of claims 1-134.

136. A pharmaceutical composition comprising:
(A) an active pharmaceutical ingredient;
(B) a co-former; and
(C) an excipient;
wherein the active pharmaceutical ingredient and the co-former forms a co-crystal, the co-crystal and the excipient are formulated into a single particle, and the co-crystal of the active pharmaceutical ingredient and the co-former have particles from about 500 nm to about 15 μm.

137. The pharmaceutical composition of claim 136, wherein the co-former comprises an acid group selected from either a phosphoric acid group, a sulfuric acid group, or carboxylic acid group.

138. The pharmaceutical composition according to either claim 136 or claim 137, wherein the co-former comprises a carboxylic acid group.

139. The pharmaceutical composition according to any one of claims 136-138, wherein the co-former comprises one or two carboxylic acid groups.

140. The pharmaceutical composition according to any one of claims 136-139, wherein the co-former is an aliphatic carboxylic acid.

141. The pharmaceutical composition according to any one of claims 136-140, wherein the co-former comprises from 1-8 carbon atoms.

142. The pharmaceutical composition according to any one of claims 136-141, wherein the co-former is an aliphatic carboxylic acid having from 3-8 carbon atoms.

143. The pharmaceutical composition according to any one of claims 136-142, wherein the co-former is an aliphatic carboxylic acid having from 3-6 carbon atoms.

144. The pharmaceutical composition according to any one of claims 136-142, wherein the co-former is an aliphatic carboxylic acid having 1 carbon atom.

145. The pharmaceutical composition of claim 144, wherein the co-former is formic acid.

146. The pharmaceutical composition according to any one of claims 136-142, wherein the co-former is an aliphatic carboxylic acid having 2 carbon atoms.

147. The pharmaceutical composition of claim 146, wherein the co-former is acetic acid or trifluoroacetic acid.

148. The pharmaceutical composition according to any one of claims 136-143, wherein the co-former is an aliphatic carboxylic acid having 4 carbon atoms.

149. The pharmaceutical composition of claim 148, wherein the co-former is succinic acid or fumaric acid.

150. The pharmaceutical composition of claim 149, wherein the co-former is succinic acid.

151. The pharmaceutical composition of claim 149, wherein the co-former is fumaric acid.

152. The pharmaceutical composition according to any one of claims 136-143, wherein the co-former is an aliphatic carboxylic acid having six carbon atoms.

153. The pharmaceutical composition of claim 152, wherein the co-former is citric acid.

154. The pharmaceutical composition according to either claim 136 or claim 137, wherein the co-former is a sulfuric acid group or phosphoric acid group.

155. The pharmaceutical composition of claim 154, wherein the inorganic acid is phosphoric acid.

156. The pharmaceutical composition of claim 136, wherein the co-former comprises a basic group.

157. The pharmaceutical composition of either claim 136 or claim 156, wherein the co-former is an aliphatic group comprising a basic group.

158. The pharmaceutical composition of claim 157, wherein the aliphatic group comprising a basic group has from 1 to 8 carbon atoms.

159. The method of either claim 157 or claim 158, wherein the aliphatic group comprising a basic group has from 3 to 8 carbon atoms.

160. The method according to any one of claims 156-159, wherein the basic group is an amine group.

161. The method according to any one of claims 156-159, wherein the basic group is a primary amine group.

162. The method according to any one of claims 156-159, wherein the basic group is a secondary amine group.

163. The method according to any one of claims 156-159, wherein the basic group is a tertiary amine group.

164. The method according to any one of claims 156-159, wherein the basic group is a quarternary amine group.

165. The pharmaceutical composition of either claim 136 or claim 137, wherein the co-former is a second active pharmaceutical ingredient.

166. The pharmaceutical composition of claim 165, wherein the second active pharmaceutical ingredient is different from the active pharmaceutical ingredient.

167. The pharmaceutical composition of either claim 165 or claim 166, wherein the second active pharmaceutical ingredient comprises an acidic group.

168. The pharmaceutical composition according to any one of claims 165-167, wherein the second active pharmaceutical ingredient comprises a carboxylic acid.

169. The pharmaceutical composition according to any one of claims 165-167, wherein the second active pharmaceutical ingredient is mycophenolic acid.

170. The pharmaceutical composition of either claim 165 or claim 166, wherein the second active pharmaceutical ingredient comprises a basic group.

171. The pharmaceutical composition according to any one of claims 165, 166, and 170, wherein the second active pharmaceutical ingredient comprises an amine group.

172. The pharmaceutical composition according to any one of claims 136-171, wherein the pharmaceutical composition comprises a molar ratio of the active pharmaceutical ingredient and co-former from about 1:5 to about 10:1.

173. The pharmaceutical composition of claim 172, wherein the molar ratio is from about 1:2 to about 5:1.

174. The pharmaceutical composition of either claim 172 or claim 173, wherein the molar ratio is from about 1:2 to about 2:1.

175. The pharmaceutical composition of claim 174, wherein the molar ratio is about 1:1.

176. The pharmaceutical composition of claim 174, wherein the molar ratio is about 2:1.

177. The pharmaceutical composition according to any one of claims 136-176, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

178. The pharmaceutical composition according to any one of claims 136-177, wherein the active pharmaceutical ingredient is an anti-inflammatory agent.

179. The pharmaceutical composition of claim 178, wherein the anti-inflammatory agent is an anti-fibrotic agent.

180. The pharmaceutical composition of either claim 178 or claim 179, wherein the anti-inflammatory agent is pirfenidone.

181. The pharmaceutical composition according to any one of claims 136-180, wherein the excipient is a sugar or sugar alcohol.

182. The pharmaceutical composition of claim 181, wherein the excipient is a sugar.

183. The pharmaceutical composition of either claim 181 or claim 182, wherein the excipient is a polysaccharide.

184. The pharmaceutical composition according to any one of claims 181-183, wherein the excipient is a disaccharide.

185. The pharmaceutical composition according to any one of claims 181-184, wherein the excipient is trehalose or lactose.

186. The pharmaceutical composition according to any one of claims 181-185, wherein the excipient is lactose.

187. The pharmaceutical composition according to any one of claims 181-186, wherein the excipient is lactose that has been milled with a particle size.

188. The pharmaceutical composition of claim 187, wherein the particle size has a D₅₀ as measured by laser diffraction from about 10 μm to about 150 μm.

189. The pharmaceutical composition of either claim 187 or claim 188, wherein the particle size has a D₅₀ as measured by laser diffraction from about 50 μm to about 125 μm.

190. The pharmaceutical composition according to any one of claims 187-189, wherein the particle size has a D₅₀ as measured by laser diffraction from about 75 μm to about 100 μm.

191. The pharmaceutical composition of claim 181, wherein the excipient is a sugar alcohol.

192. The pharmaceutical composition of claim 191, wherein the excipient is mannitol.

193. The pharmaceutical composition according to any one of claims 136-180, wherein the excipient is an amino acid or a peptide.

194. The pharmaceutical composition of claim 193, wherein the excipient is an amino acid.

195. The pharmaceutical composition according to any one of claim 193 or 194, wherein the excipient is a hydrophobic amino acid.

196. The pharmaceutical composition according to any one of claims 193-195, wherein the excipient is an aliphatic hydrophobic amino acid.

197. The pharmaceutical composition according to any one of claims 193-196, wherein the excipient is leucine.

198. The pharmaceutical composition according to any one of claim 193, or 194, wherein the excipient is a polar amino acid.

199. The pharmaceutical composition according to any one of claim 193, 194, or 198, wherein the excipient is lysine.

200. The pharmaceutical composition according to any one of claim 193 or claim 194,
wherein the excipient is glycine.

201. The pharmaceutical composition according to any one of claims 136-200, wherein the pharmaceutical composition comprises particles of the co-crystal having a size from about 250 nm to about 10 μm.

202. The pharmaceutical composition of claim 201, wherein the size of the particles is from about 1.25 μm to about 7.5 μm.

203. The pharmaceutical composition of either claim 201 or claim 202, wherein the size of the particles is from about 1.5 μm to about 6 μm.

204. The pharmaceutical composition according to any one of claims 201-203, wherein the size of the particles is from 2 μm to about 3 μm.

205. The pharmaceutical composition according to any one of claims 136-204, wherein the pharmaceutical composition has a mass median aerodynamic diameter (MMAD) from about 1.0 μm to about 8.0 μm.

206. The pharmaceutical composition of claim 205, wherein the MMAD is from about 1.5 μm to about 6.0 μm.

207. The pharmaceutical composition of either claim 205 or claim 206, wherein the MMAD is from about 1.75 μm to about 4.0 μm.

208. The pharmaceutical composition according to any one of claims 136-207, wherein the pharmaceutical composition has a geometric standard deviation (GSD) from about 1.0 to about 8.0.

209. The pharmaceutical composition of claim 208, wherein the GSD is from about 1.25 to about 6.0.

210. The pharmaceutical composition of either claim 208 or claim 209, wherein the GSD is from about 1.5 to about 4.0.

211. The pharmaceutical composition according to any one of claims 136-210, wherein the pharmaceutical composition has a fine powder fraction of the recovered dose of greater than 30%.

212. The pharmaceutical composition of claim 211, wherein the fine powder fraction of the recovered dose is greater than 40%.

213. The pharmaceutical composition of either claim 211 or claim 212, wherein the fine powder fraction of the recovered dose is greater than 50%.

214. The pharmaceutical composition according to any one of claims 136-213, wherein the pharmaceutical composition has an emitted dose of the recovered dose of greater than 70%.

215. The pharmaceutical composition of claim 214, wherein the emitted dose of the recovered dose is greater than 80%.

216. The pharmaceutical composition of either claim 214 or claim 215, wherein the emitted dose of the recovered dose is greater than 85%.

217. The pharmaceutical composition according to any one of claims 136-216 comprising:
(A) pirfenidone;
(B) a co-former; wherein the co-former is a C1-C8 aliphatic carboxylic acid; and
(C) an excipient, wherein the excipient is mannitol, leucine, or lactose.

218. The pharmaceutical composition according to any one of claims 136-217, wherein the pharmaceutical composition is formulated for administration via injection, orally, or via inhalation.

219. The pharmaceutical composition according to any one of claims 136-218, wherein the pharmaceutical composition is formulated for administration via inhalation.

220. The pharmaceutical composition according to any one of claims 136-219, wherein the pharmaceutical composition is formulated as a unit dose.

221. The pharmaceutical composition according to any one of claims 136-220, wherein the pharmaceutical composition is formulated for use with an inhaler.

222. The pharmaceutical composition of claim 221, wherein the inhaler is a fixed dose combination inhaler, a single dose dry powder inhaler, a multi-dose dry powder inhaler, multi-unit dose dry powder inhaler, a metered dose inhaler, or a pressurized metered dose inhaler.

223. The pharmaceutical composition of claim 222, wherein the inhaler is a capsule-based inhaler.

224. The pharmaceutical composition according to any one of claims 221-223, wherein the inhaler is a low resistance inhaler.

225. The pharmaceutical composition according to any one of claims 221-223, wherein the inhaler is a high resistance inhaler.

226. The pharmaceutical composition according to any one of claims 221-225, wherein the inhaler is used with a flow rate from about 10 L/min to about 150 L/min.

227. The pharmaceutical composition of claim 226, wherein the flow rate is from about 20 L/min to about 100 L/min.

228. The pharmaceutical composition according to any one of claims 221-227, wherein the inhaler has a loaded dose from about 0.1 mg to about 500 mg.

229. The pharmaceutical composition of claim 228, wherein the inhaler has a loaded dose from about 0.1 mg to about 100 mg.

230. The pharmaceutical composition of claim 228, wherein the inhaler has a loaded dose from about 50 mg to about 500 mg.

231. The pharmaceutical composition of claim 54, wherein the loaded dose is from about 5 mg to about 25 mg.

232. The pharmaceutical composition according to any one of claims 221-231, wherein the inhaler is configured to deliver one or a series of doses from one or more unit doses loaded sequentially.

233. The pharmaceutical composition of claim 232, wherein the inhaler is configured to deliver one dose from one unit dose.

234. The pharmaceutical composition of claim 232, wherein the inhaler is configured to deliver a series of doses from one unit dose.

235. The pharmaceutical composition of claim 232, wherein the inhaler is configured to deliver one dose each from a series of capsules loaded sequentially.

236. The pharmaceutical composition of claim 232, wherein the inhaler is configured to deliver a series of doses from a series of capsules loaded sequentially.

237. A method of treating a disease or disorder comprising administering to the patient in need thereof a therapeutically effective amount of the pharmaceutical composition according to any one of claims 136-236, wherein the active pharmaceutical ingredient is useful to treating the disease or disorder.

238. A method of preventing a disease or disorder comprising administering to the patient in need thereof a therapeutically effective amount of the pharmaceutical composition according to any one of claims 136-236, wherein the active pharmaceutical ingredient is useful to prevent the disease or disorder.

239. A kit comprising:
(A) a pharmaceutical composition according to any one of claims 136-236;
(B) a capsule comprising a unit dose of the pharmaceutical composition, a blister pack comprising a unit dose of the pharmaceutical composition, or a metering device that distributes a unit dose of the pharmaceutical composition; and
(C) an aerosolizing device that disperses the unit dose.

240. The kit of claim 239, wherein the aerosolizing device is an inhaler.

241. The kit of either claim 239 or claim 240 comprising a capsule comprising a unit dose of the pharmaceutical composition.

242. The kit of either claim 239 or claim 240 comprising a blister pack comprising a unit dose of the pharmaceutical composition.

243. The kit of either claim 239 or claim 240 comprising a metering device that distributes a unit dose of the pharmaceutical composition.
